SEQUENCE LISTING (9 sequences)

SEQ ID NO: 1 - DNA, 1810 bp, Aeromonas hydrophila
  5′UTR (1)..(480)
  CDS (481)..(1602)
  sig_peptide (481)..(540)
  mat_peptide (541)..(1602)
  3′UTR (1603)..(1810)
gtaatacgactcactatagggcgaattgggtacacttacctggtaccccacccgggtggaaaatcgatgggcccgcggccgctctagaagtactctcgagaagctttttgaattctttggatcctcggcgtgggccatggcctgttcggcgggttcgctctgccagcggttggggccgaggtgatccgacctcttcttctatttataaggcgagtcgtcgttattgtgtgataaatcaccaattcggacgaattttgccagcggttatcgctgtaaacgttttcccatggcgtgcaaacaatgtgggattcaggtcacaatttttccgctgtgactatgcttttcgtaaaaagttccaagttttttcattgcggattggaaaacccggtgctagtctcggcgccatagtgatgcaaagtacatcgctaacacagggaataacaacgacttagtgtttaattacagtaggcattggaaactatgaaaaagacaattctggctattgctatcccggctctgtttgcatccgccgctaacgctgcagtggtttacgacaaagacggtaccacttttgacgtatacggccgtgttcaggctaactactacggtgaccacaacaaatctgtagctgctaccgatggttcctggggcttcagcggaactggtaccccggaatatactcctggtaccgctgctcattactctgatgttgatggtgagctggttggttcttcccgtctgggttggtccggtaagattgccctgaacaacacctggtccggtatcgccaagactgagtggcaagtttctgctgaaaactccgccaacaagttcgattcccgtcacatctacgttggtttcgacggcacccagtacggtaagatcatcttcggtcagaccgataccgcgttctatgacgtgctggaaccgaccgatatcttcaacgagtggggcgacgtaggtaacttctatgacggtcgtcaagaaggtcagatcatctactccaacacctacggtggcttcaaaggcaaactgtcctatcaaaccaacgacgacaaggccgtcaaggttactgacgtaggtcagggcatcaaagaaaacgcagtgtacggcaaggatgttaagcgtaactacggttatgccgcggctgccggttatgacttcgacttcggtctgggtctgaacgcaggttactcctactccgatctggaaaataccgcaaccaacaacactggcgacaagaaagagtgggcactgggtgcacactacgccatcaacggtttctacttcgccggtgtctacacccaggcagatctgagctatgacaccaccaccggtggtgacaaggacaagggccgtggctacgagctggctgcttcctacaacgttgatgcctggactttcctggccggctacaacttcactgaaggtaaagttgcttccaacaccgctggtgctgagtacaaagacatcgttgacgaaaccctgctgggcgtacagtacgctttcacttccaagctgaaagcctacaccgagtacaagatccagggtatcgacaagatggacgacgagtggaccgttgccctgcaatacaacttctaatctagcctctgcgttgatttagatgatgaacggccaagcttgcttggccgttttgttttatctgcttcccacctgatgtttctgttctcttctgttgattatcttctccttgccctctttgacttgcgtcagttcacgttgtctcttttctgtacttggctcccgggcagcggatcgctagattattcagctcgttgcaggatgtaaa

SEQ ID NO: 2 - protein, 373 aa, Aeromonas hydrophila
MKKTILAIAIPALFASAANAAVVYDKDGTTFDVYGRVQANYYGDHNKSVAATDGSWGFSGTGTPEYTPGTAAHYSDVDGELVGSSRLGWSGKIALNNTWSGIAKTEWQVSAENSANKFDSRHIYVGFDGTQYGKIIFGQTDTAFYDVLEPTDIFNEWGDVGNFYDGRQEGQIIYSNTYGGFKGKLSYQTNDDKAVKVTDVGQGIKENAVYGKDVKRNYGYAAAAGYDFDFGLGLNAGYSYSDLENTATNNTGDKKEWALGAHYAINGFYFAGVYTQADLSYDTTTGGDKDKGRGYELAASYNVDAWTFLAGYNFTEGKVASNTAGAEYKDIVDETLLGVQYAFTSKLKAYTEYKIQGIDKMDDEWTVALQYNF

SEQ ID NO: 3 - DNA, 1122 bp, Aeromonas hydrophila
  CDS (1)..(1122)
  sig_peptide (1)..(60)
  mat_peptide (61)..(1122)
atgaaaaagacaattctggctattgctatcccggctctgtttgcatccgccgctaacgctgcagtggtttacgacaaagacggtaccacttttgacgtatacggccgtgttcaggctaactactacggtgaccacaacaaatctgtagctgctaccgatggttcctggggcttcagcggaactggtaccccggaatatactcctggtaccgctgctcattactctgatgttgatggtgagctggttggttcttcccgtctgggttggtccggtaagattgccctgaacaacacctggtccggtatcgccaagactgagtggcaagtttctgctgaaaactccgccaacaagttcgattcccgtcacatctacgttggtttcgacggcacccagtacggtaagatcatcttcggtcagaccgataccgcgttctatgacgtgctggaaccgaccgatatcttcaacgagtggggcgacgtaggtaacttctatgacggtcgtcaagaaggtcagatcatctactccaacacctacggtggcttcaaaggcaaactgtcctatcaaaccaacgacgacaaggccgtcaaggttactgacgtaggtcagggcatcaaagaaaacgcagtgtacggcaaggatgttaagcgtaactacggttatgccgcggctgccggttatgacttcgacttcggtctgggtctgaacgcaggttactcctactccgatctggaaaataccgcaaccaacaacactggcgacaagaaagagtgggcactgggtgcacactacgccatcaacggtttctacttcgccggtgtctacacccaggcagatctgagctatgacaccaccaccggtggtgacaaggacaagggccgtggctacgagctggctgcttcctacaacgttgatgcctggactttcctggccggctacaacttcactgaaggtaaagttgcttccaacaccgctggtgctgagtacaaagacatcgttgacgaaaccctgctgggcgtacagtacgctttcacttccaagctgaaagcctacaccgagtacaagatccagggtatcgacaagatggacgacgagtggaccgttgccctgcaatacaacttctaa

SEQ ID NO: 4 - protein, 373 aa, Aeromonas hydrophila; identical to SEQ ID NO: 2

SEQ ID NO: 5 - DNA, 60 bp, Aeromonas hydrophila
  CDS (1)..(60)
atgaaaaagacaattctggctattgctatcccggctctgtttgcatccgccgctaacgct

SEQ ID NO: 6 - protein, 20 aa, Aeromonas hydrophila
MKKTILAIAIPALFASAANA

SEQ ID NO: 7 - DNA, 1062 bp, Aeromonas hydrophila
  CDS (1)..(1062)
gcagtggtttacgacaaagacggtaccacttttgacgtatacggccgtgttcaggctaactactacggtgaccacaacaaatctgtagctgctaccgatggttcctggggcttcagcggaactggtaccccggaatatactcctggtaccgctgctcattactctgatgttgatggtgagctggttggttcttcccgtctgggttggtccggtaagattgccctgaacaacacctggtccggtatcgccaagactgagtggcaagtttctgctgaaaactccgccaacaagttcgattcccgtcacatctacgttggtttcgacggcacccagtacggtaagatcatcttcggtcagaccgataccgcgttctatgacgtgctggaaccgaccgatatcttcaacgagtggggcgacgtaggtaacttctatgacggtcgtcaagaaggtcagatcatctactccaacacctacggtggcttcaaaggcaaactgtcctatcaaaccaacgacgacaaggccgtcaaggttactgacgtaggtcagggcatcaaagaaaacgcagtgtacggcaaggatgttaagcgtaactacggttatgccgcggctgccggttatgacttcgacttcggtctgggtctgaacgcaggttactcctactccgatctggaaaataccgcaaccaacaacactggcgacaagaaagagtgggcactgggtgcacactacgccatcaacggtttctacttcgccggtgtctacacccaggcagatctgagctatgacaccaccaccggtggtgacaaggacaagggccgtggctacgagctggctgcttcctacaacgttgatgcctggactttcctggccggctacaacttcactgaaggtaaagttgcttccaacaccgctggtgctgagtacaaagacatcgttgacgaaaccctgctgggcgtacagtacgctttcacttccaagctgaaagcctacaccgagtacaagatccagggtatcgacaagatggacgacgagtggaccgttgccctgcaatacaacttctaa

SEQ ID NO: 8 - protein, 353 aa, Aeromonas hydrophila
AVVYDKDGTTFDVYGRVQANYYGDHNKSVAATDGSWGFSGTGTPEYTPGTAAHYSDVDGELVGSSRLGWSGKIALNNTWSGIAKTEWQVSAENSANKFDSRHIYVGFDGTQYGKIIFGQTDTAFYDVLEPTDIFNEWGDVGNFYDGRQEGQIIYSNTYGGFKGKLSYQTNDDKAVKVTDVGQGIKENAVYGKDVKRNYGYAAAAGYDFDFGLGLNAGYSYSDLENTATNNTGDKKEWALGAHYAINGFYFAGVYTQADLSYDTTTGGDKDKGRGYELAASYNVDAWTFLAGYNFTEGKVASNTAGAEYKDIVDETLLGVQYAFTSKLKAYTEYKIQGIDKMDDEWTVALQYNF

SEQ ID NO: 9 - protein, 20 aa, Aeromonas hydrophila
AVFYDKDGTTFDVYGRVQAN

(19) United States
(12) Patent Application Publication
(10) Pub. No.: US 2004/0077067 A1
(43) Pub. Date: Apr. 22, 2004

(54) Therapeutic and prophylactic agents derived from aeromonas hydrophila bacterial surface proteins

(76) Inventors: Yoke Min Sin, Singapore (SG); Ruowen Ge, Singapore (SG); Haoming Fang, Singapore (SG)

Correspondence Address: Klarquist Sparkman Campbell, Leigh & Whinston, One World Trade Center Suite 1600, 121 S W Salmon Street, Portland, OR, 97204, US

(21) Appl. No.: 10/220,986

(22) PCT Filed: Mar. 7, 2001
(86) PCT No.: PCT/SG01/00029

(30) Foreign Application Priority Data
Mar. 8, 2000 (SG) 200001261-7

(51) Int. Cl. C12N 1/20; C07K 1/00; C07K 14/00; C07K 17/00

(52) U.S. Cl. 435/252.3

(57) ABSTRACT

The invention provides a novel surface polypeptide from Aeromonas hydrophila as well as fragments, variants and derivatives of this polypeptide. Also provided are polynucleotides encoding the polypeptide, fragments, variants and derivatives. Compositions containing the polypeptide and polynucleotides of the invention are also disclosed as well as methods useful in the treatment and prevention of bacterial infection in an animal, wherein said infection is caused by bacteria of a genus selected from the group consisting of Aeromonas, Vibrio and Edwardsiella, and in the diagnosis of bacterial infection in an animal, wherein said infection is caused by bacteria of a genus Aeromonas.

### FIELD OF THE INVENTION

This invention relates, in general, to bacterial surface proteins. More particularly, the present invention relates to polynucleotides from Aeromonas, particularly Aeromonas hydrophila, which encode a novel recombinant adhesin or a biologically active fragment thereof, or a variant or derivative of these and to expression vectors comprising such polynucleotides operably linked to regulatory nucleic acids. The invention further relates to recombinant adhesin polypeptides, fragments of such polypeptides, as well as variants and derivatives of these. The invention also extends to antibodies to recombinant polypeptides and to the use of the polynucleotide, polypeptides and antibodies of the invention inter alia for diagnostic purposes and for treatment and prevention of infection by Aeromonas species and related bacteria.

### BACKGROUND OF THE INVENTION

Aeromonas hydrophila is a gram-negative bacterium that infects a wide range of hosts including amphibian, reptilian and avian species as well as mammals such as cows and humans (Popoff, M. Aeromonas. In: Bergy 's Manual of Systematic Bacteriology, N. R. Krieg (ed), Williams & Wilkins, Baltimore, Md., 1984, vol. I, pp. 545-548), but it is most well-known as a pathogen of marine animals such as fish. It causes motile aeromonad septicemia (MAS), which results in great economic losses in freshwater fish farming. Antibiotics are often used for prevention and treatment of MAS (Stevenson, RWM. “Vaccination against Aeromonas hydrophila.” In: Fish Vaccination. Ellis, A E (ed), Academic Press, London, 1988, pp 112-123). However, extensive use of antibiotics has serious drawback of increasing plasmid-coding antibiotic resistance in A. hydrophila. As A. hydrophila is also an opportunistic human pathogen, antibiotic-resistant strains may become a health problem. Hence, a vaccine against A. hydrophila is an attractive option to prevent the occurrence of MAS. In this respect, there are some severe fish diseases that have been promisingly controlled by various vaccines, including:

1. Furunculosis, which is an important disease of wild and farmed salmonids throughout the world caused by Aeromonas salmonicida, has been successfully controlled by using a vaccine comprising bacterins and oil-based adjuvants (Midtlyng, P J. “Vaccination against furunculosis.” In: Furunculosis in Fish: A multidisciplinary Review. Bemoth E M, Ellis A E, Midtlyng P J, Oliver G, Smith P (eds), Academic Press, London, 1997, pp 382-404);

2. Yersiniosis (also called enteric redmouth disease (ERM)), another important salmonid fish disease caused by Yersinia ruckeri, can be controlled by bacterin which provides cross-protection among serogroups of Yersinia ruckeri (Stevenson, RMW. “Immunization with bacterial antigens: Yersiniosis.” In: Fish Vaccinology. Gudding R, Lillehaug A, Midtlyng P J, Brown F (eds), Developments in Biological Standardization. Basel, Karger, 1997, vol 90, pp 117-124);

3. Edwardsiellosis (also called enteric septicaemia of catfish (ESC)) is the most severe disease affecting commercial catfish culture in the United States. Two vaccines have been produced to vaccinate catfish against Edwardsiella ictaluri (Thune, et al., “Immunization with bacterial antigens: Edwardsiellosis.” In: Fish Vaccinology. Gudding R, Lillehaug A, Midtlyng P J, Brown F (eds), Developments in Biological Standardization. Basel, Karger, 1997, vol 90, pp 125-134); and

4. Vibriosis is the most economically important disease in marine fish culture caused by several species of Vibrio. Many commercial vaccines have been developed against one or several species of Vibrio (Toranzo, et al., “Immunization with bacterial antigens: Vibrio infections.” In: Fish Vaccinology. Gudding R, Lillehaug A, Midtlyng P J, Brown F (eds), Developments in Biological Standardization. Basel, Karger, 1997, vol 90, pp 93-105).

Inspired by the successful development of vaccines against various fish pathogens, there have been attempts to use attenuated preparations of A. hydrophila or extracellular products of this organism to immunise fish against A. hydrophila infection. However, due to the antigenic diversity of A. hydrophila strains, it has been difficult to develop a useful vaccine and accordingly, there are no effective vaccines currently known or commercially available for protection against A. hydrophila.

In work leading up to the present invention, the present inventors identified a 43-kDa outer membrane protein as an important adhesin of A. hydrophila strain PPD 134/91 (Lee, et al., 1997, Journal of Fish Diseases 20: 169-175). N-terminal sequence analysis of this protein revealed a 20 residue sequence with substantial homology to the 39-kDa outer membrane protein, Omp II, from A. hydrophila Ah 65 isolated from rainbow trout by Jeanteaur et al. (1992, Mol. Microbiol. 6: 3355-3363), and to the 40-kDa pore-forming carbohydrate-reactive outer membrane protein (CROMP) isolated from the human isolate A. hydrophila A6 by Quinn et al. (1994, infection and Immunity 62: 4054-4058). Crude preparations of the A. hydrophila PPD 134/91 adhesin were shown in vitro to cross-inhibit serologically different species of A. hydrophila, Aeromonas sobria or some virulent strains of Vibrio spp. from invading into cpithelioma papillosum of carp (EPC) cells (Fang H M, Ling K C, Tan Y L, Ge R and Sin Y M (1998). “In vitro inhibition of epithelial cell invasion by Aeromonas hydrophila and Vibrio species by fish Aeromonas hydrophila major adhesin” Journal of Fish Diseases. 21, 273-280).

### SUMMARY OF THE INVENTION

The present invention arises at least in part from the discovery of full-length nucleotide and polypeptide sequences relating to a 43-kDa adhesin of Aeromonas and more particularly of A. hydrophila. The discovery of these sequences, thus, allows for the first time the production by recombinant techniques of large quantities of this adhesin and its biologically active fragments as well as variants and derivatives of these. It has also been found that this adhesin can elicit a protective or therapeutic immune response against infection by Aeromonas, Vibrio and Edwardsiella species.

Accordingly, in one aspect of the present invention, there is provided a recombinant polypeptide comprising the sequence set forth in SEQ ID NO: 2, 4 or 8, or fragment thereof, or variant or derivative of these with the proviso that said fragment does not consist of the sequence set forth in SEQ ID NO: 9.

The recombinant polypeptide may comprise a leader peptide. Suitably, the leader peptide comprises the sequence set forth in SEQ ID NO: 6, or a fragment thereof, or variant or derivative of these. In such a case, the recombinant polypeptide preferably comprises the sequence set forth in SEQ ID NO: 2 or 4.

In another aspect, the invention provides an isolated polynucleotide encoding a polypeptide, fragment, variant or derivative as broadly described above. Preferably, the polynucleotide comprises the sequence set forth in SEQ ID NO: 7, or a fragment thereof, or a polynucleotide variant of these.

The polynucleotide preferably comprises a nucleotide sequence encoding a leader peptide. Suitably, said nucleotide sequence comprises the sequence set forth in SEQ ID NO: 5 or a fragment thereof, or a polynucleotide variant of these. In such a case, the polynucleotide preferably comprises the sequence set forth in SEQ ID NO: 1 or 3.

Preferably, the variant is obtained from a bacterial species. Preferably, the bacterial species is of a genus Aeromonas.

In another aspect, the invention features an expression vector comprising a polynucleotide as broadly described above wherein the polynucleotide is operably linked to one or more regulatory nucleic acids.

In a further aspect, the invention provides a host cell containing said expression vector.

The invention also contemplates a method of producing a recombinant polypeptide, fragment, variant or derivative as broadly described above, comprising:

(a) culturing a host cell containing an expression vector as broadly described above such that said recombinant polypeptide, fragment, variant or derivative is expressed from said polynucleotide; and

(b) isolating the recombinant polypeptide, fragment, variant or derivative.

In a further aspect, the invention provides a method of producing an immuno-interactive fragment of a polypeptide as broadly described above, comprising:

(a) administering a fragment of said polypeptide to an animal; and

(b) detecting an immune response in said animal, including the production of elements that protect said animal against infection by a bacterial species of a genus selected from the group consisting of Aeromonas, Vibrio and Edwardsiella or that specifically bind to said polypeptide or to said bacterial species, which is indicative of said fragment being said immuno-interactive fragment.

In another aspect, the invention provides a method of producing an immuno-interactive fragment of a polypeptide as broadly described above, comprising:

(a) combining a fragment of said polypeptide with at least one antigen-binding molecule that binds to said polypeptide as broadly described above; and

(b) detecting the presence of a conjugate comprising said fragment and said antigen-binding molecule, which is indicative of said fragment being a said immuno-interactive fragment.

According to another aspect, the invention provides a method of producing a variant of a polypeptide as broadly described above, or of an immuno-interactive fragment of said polypeptide, said method comprising:

(a) administering a test polypeptide suspected of being said variant to an animal, wherein said test polypeptide is distinguished from said polypeptide or said immuno-interactive fragment by substitution of at least one amino acid with a different amino acid; and

(b) detecting an immune response in said animal, including the production of elements that protect said animal against infection by a bacterial species of a genus selected from the group consisting of Aeromonas, Vibrio and Edwardsiella or that specifically bind to said polypeptide or to said bacterial species, which is indicative of said test polypeptide being a said variant.

In yet another aspect, the invention contemplates a method of producing a variant of a polypeptide as broadly described above, or of an immuno-interactive fragment of said polypeptide, said method comprising:

(a) combining a test polypeptide suspected of being said variant with at least one antigen-binding molecule that binds to a said polypeptide or immuno-interactive fragment as broadly described above, wherein said test polypeptide is distinguished from said polypeptide or said immuno-interactive fragment by substitution of at least one amino acid with a different amino acid; and

(b) detecting the presence of a conjugate comprising said test polypeptide and said antigen-binding molecule, which is indicative of said test polypeptide being a said variant.

In another aspect, the invention provides a composition which may be used to elicit an immune response in an animal, including the production of elements that protect said animal against infection by a bacterial species of a genus selected from the group consisting of Aeromonas, Vibrio and Edwardsiella or that specifically bind to said polypeptide or to said bacterial species, comprising a polypeptide, variant or derivative as broadly described above and a pharmaceutically acceptable carrier.

Optionally, said composition further comprises an adjuvant.

In yet another aspect of the invention there is provided a method for eliciting an immune response in an animal, comprising administering to said animal an immunogenically effective amount of a composition as broadly described above.

Suitably, said animal is a freshwater and/or marine animal, preferably a fish.

Preferably, said infection is associated with fish motile aeromonad septicemia, Vibriosis and Edwardsiellosis.

In another aspect, the invention resides in the use of a recombinant polypeptide, fragment, variant or derivative according to the present invention to produce an antigen-binding molecule that binds specifically to the recombinant polypeptide, fragment, variant or derivative.

In yet another aspect, the invention provides antigen-binding molecules, including antibodies so produced.

The invention also extends to a method of detecting in a sample a polypeptide, fragment, variant or derivative as broadly described above, comprising:

(a) contacting the sample with an antigen-binding molecule as broadly described above; and

(b) detecting the presence of a complex comprising said antigen-binding molecule and said polypeptide, fragment, variant or derivative in said contacted sample.

According to a further aspect, there is provided a method of detecting bacteria, preferably of a genus selected from the group consisting of Aeromonas Vibrio, Edwardsiella, in a biological sample suspected of containing said bacteria, said method comprising:

(a) isolating the biological sample from an animal;

(b) detecting a polynucleotide sequence as broadly described above in said sample, which indicates the presence of said bacteria.

The invention further contemplates a method for detecting or diagnosing infection of animals by bacteria comprising:

(a) contacting a biological sample from an animal with a polypeptide, fragment, variant or derivative of the invention; and

(b) determining the presence or absence of a complex between said polypeptide, fragment, variant or derivative and antibodies in said sample, wherein the presence of said complex is indicative of said infection.

In one embodiment, the bacterial infection detected or diagnosed is of a genus selected from the group consisting of Aeromonas Vibrio, Edwardsiella.

The invention also extends to the use of the polypeptide, nucleic acids, and antigen binding molecule, including antibody of the present invention in a kit for detecting or diagnosing bacterial infenction in a biological sample. In one embodiment, the bacteria detected are of a genus selected from the group consisting of Aeromonas, Vibrio, Edwardsiella.

The invention also provides oligonucleotides which specifically hybridize to the polynucleotides of the invention or their complement.

### BRIEF DESCRIPTION OF THE DRAWINGS

FIG. 1 is an illustration of the gene-cloning scheme used to produce the recombinant adhesin. In panel A, two degenerate primer P1 and P2 are shown, which were designed according to N-terminal amino acids of the 43 kDa protein for PCR reaction. After PCR, one 59 bp dominant DNA fragment was subcloned (plasmid pT1) and sequenced. The amino acid sequence deduced from the 59 bp fragment of the plasmid pT1 corresponded to the N-terminal sequence of the known 43 kDa protein. A specific sense primer SP3 was constructed according to the central region of this 59 bp fragment and used for 3′ nested RACE PCR using genomic library 1 as template. Panel B shows the strategy of cloning the full length gene encoding the 43-kDa adhesin. The coding region is boxed. Specific sense primers SP3, FS20 and FS21 were used for 3′RACE PCR, and specific anti-sense primers FAS3 and FAS1 were used for 5′RACE PCR. Different cloned fragments are shown and the full-length gene was cloned in pTAH.

FIG. 2 shows the nucleotide sequence of the cloned gene which has an open reading frame (OPF) of 1119 bp encoding a predicted precursor protein of 373 amino acids containing a putative 20 amino acid signal peptide (underlined). The mature major adhesin protein is predicted to comprise 353 amino acids (Mr 38.7 kDa). The 20 amino acid N-terminal sequence disclosed in Lee et al (1997, supra) is in bold typeface. The start and stop codons are also shown in bold typeface.

FIG. 3 is an image of an SDS PAGE gel showing bacterial expression of the recombinant adhesin. Lane 1: Molecular weight markers (Bio-Rad), apparent molecular weight is shown in kDa. Lanes 2 and 3: show total lysates of E. coli M15 transformed with pQE-ahma. In lane 2, cells were not induced with IPTG, and in lane 3, protein expression was induced with IPTG. Lane 4: a substantially pure recombinant major adhesin protein obtained by Ni-NTA affinity chromatography. Samples were boiled for 5 min in the presence of β-mercaptoethanol, resolved by 12% SDS-PAGE and stained with 0.125% CBB.

FIG. 4 is an image of an immunoblot of whole cell lysates of various bacteria against a polyclonal antisera raised against the 43-kDa recombinant adhesin. Lanes: 1, molecular weight markers in kDa; 2, recombinant adhesin; 3, A. hydrophila PPD 134/91; 4, A. hydrophila L31; 5, A. hydrophila PPD 11/90; 6, A. hydrophila AH 94063; 7, A. hydrophila PPD 122/91; 8, A. hydrophila ST-78-3-3; 9, A. hydrophila PPD 70/91; 10, A. hydrophila ATCC 7966; 11, A. sobria CR-79-1-1; 12, A. hydrophila PPD 35/85 (avirulent); 13, V. anguillarum 01/10/93(2); 14, V. anguillarum 811218-5W; 15, E. tarda 130/90.

FIG. 5 is a graph showing changes in the agglutinating antibody titers in the serum of blue gourami after intraperitoneal injection of 15 μg recombinant adhesin in FCA. For control, blue gourami were injected with PBS and FCA only.

### DETAILED DESCRIPTION OF THE INVENTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

“Amplification product” refers to a nucleic acid product generated by nucleic acid amplification techniques.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

As used herein, the term “binds specifically” and the like refers to antigen-binding molecules that bind the polypeptide or polypeptide fragments of the invention.

The term “biological sample” as used herein refers to a sample that may be extracted, untreated, treated, diluted or concentrated from an animal. The biological sample may be selected from the group consisting of whole blood, serum, plasma, saliva, urine, sweat, ascitic fluid, peritoneal fluid, synovial fluid, amniotic fluid, cerebrospinal fluid, skin biopsy, and the like. The biological sample preferably includes serum, whole blood, plasma, and lymph as well as other circulatory fluid and saliva, mucus secretion and respiratory fluid. More preferably, the biological sample is a circulatory fluid such as serum or whole blood or a fractionated portion thereof. Most preferably, the biological sample is serum or a fractionated portion thereof.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “corresponds to” or “corresponding to” is meant a polynucleotide (a) having a nucleotide sequence that is substantially identical or complementary to all or a portion of a reference polynucleotide sequence or (b) encoding an amino acid sequence identical to an amino acid sequence in a peptide or protein. This phrase also includes within its scope a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties or by post-translational modification techniques as would be understood in the art. The term “derivative” also includes within its scope alterations that have been made to a parent sequence including addition, or deletion of at least one amino acid that provide for functional equivalent molecules. Accordingly, the term derivative encompasses molecules that will elicit an immune response, including the production of elements that protect an animal against infection by a bacterial species or that specifically bind to a polypeptide of the invention or to said bacterial species.

For the purposes of the present invention, the phrase “elicit(s) an immune response” refers to the ability of a polypeptide or immuno-interactive fragment or variant derivative, or a polynucleotide of the invention to produce an immune response in an animal to which it is administered, including the production of elements that protect the animal against infection by a bacterial species or that specifically bind to a polypeptide of the invention or to said bacterial species.

By “expression vector” is meant any autonomous genetic element capable of directing the synthesis of a protein encoded by the vector. Such expression vectors are known by practitioners in the art.

As used herein, the term “function” refers to a biological, enzymatic, or therapeutic function.

“Homology” refers to the percentage number of amino acids that are identical or constitute conservative substitutions as defined in Table A below. Homology may be determined using sequence comparison programs such as GAP (Deveraux et al. 1984, Nucleic Acids Research 12, 387-395). In this way, sequences of a similar or substantially different length to those cited herein might be compared by insertion of gaps into the alignment, such gaps being determined, for example, by the comparison algorithm used by GAP.

By “immunologically effective amount” is meant the administration to an animal of an amount of a polypeptide, immuno-interactive fragment, variant or derivative or a polynucleotide of the invention, either in a single dose or as part of a series, that is effective for raising an immune response against that polypeptide, immuno-interactive fragment, variant or derivative or the polypeptide encoded by that polynucleotide against a bacterium comprising said polypeptide, immuno-interactive fragment, variant or derivative. The effective amount will vary depending upon the taxonomic group of animal to be treated, the capacity of the animal's immune system to elicit an immune response (inclusive of a humoral and/or a cellular immune response), and the formulation of the vaccine. It is expected that the amount will fall in a relatively broad range that can be determined through routine trials.

Reference herein to “immuno-interactive” includes reference to any interaction, reaction, or other form of association between molecules and in particular where one of the molecules is, or mimics, a component of the immune system.

By “immuno-interactive fragment” is meant a fragment of a polypeptide comprising the sequence in any one of SEQ ID NO: 2, 4 or 8 which fragment elicits an immune response, including the production of elements that specifically bind to said polypeptide, or variant or derivative thereof or against a bacterial species comprising said polypeptide, variant or derivative. As used herein, the term “immuno-interactive fragment” includes deletion mutants and small peptides, for example of at least six, preferably at least 8 and more preferably at least 20 contiguous amino acids, which comprise antigenic determinants or epitopes. Several such fragments may be joined together. Peptides of this type may be obtained through the application of standard recombinant nucleic acid techniques or synthesised using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines ” edited by Nicholson and published by Blackwell Scientific Publications. Alternatively, peptides can be produced by digestion of a polypeptide of the invention with proteinases such as endoLys-C, endoArg-C, endoGlu-C and staphylococcus V8-protease. The digested fragments can be purified by, for example, high performance liquid chromatographic (HPLC) techniques.

By “isolated” is meant material that is substantially or essentially free from components that normally accompany it in its native state. For example, an “isolated polynucleotide”, as used herein, refers to a polynucleotide, which has been purified from the sequences which flank it in a naturally occurring state, e.g., a DNA fragment which has been removed from the sequences which are normally adjacent to the fragment.

By “natural gene” is meant a gene that naturally encodes the protein. However, it is possible that the parent polynucleotide encodes a protein that is not naturally occurring but has been engineered using recombinant techniques.

By “obtained from” is meant that a sample such as, for example, a polynucleotide extract or polypeptide extract is isolated from, or derived from, a particular source of the host. For example, the extract can be obtained from a tissue or a biological fluid isolated directly from the host.

The term “oligonucleotide” as used herein refers to a polymer composed of a multiplicity of nucleotide residues (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length, generally from about 10 to 30 nucleotide residues, but the term can refer to molecules of any length, although the term “polynucleotide” or “nucleic acid” is typically used for large oligonucleotides.

By “operably linked” is meant that transcriptional and translational regulatory polynucleotides are positioned relative to a polypeptide-encoding polynucleotide in such a manner that the polynucleotide is transcribed and the polypeptide is translated.

By “pharmaceutically-acceptable carrier” is meant a solid or liquid filler, diluent or encapsulating substance that can be safely used in topical or systemic administration to a mammal.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally occurring amino acid polymers.

The term “polynucleotide” or “nucleic acid” as used herein designates mRNA, RNA, cRNA, cDNA or DNA. The term typically refers to oligonucleotides greater than 30 nucleotide residues in length.

The terms “polynucleotide variant” refers to polynucleotides displaying at least 70% sequence identity with a reference polynucleotide sequence, or polynucleotides that hybridise with a reference sequence under stringent conditions that are defined hereinafter. The term also encompasses polynucleotides with one or more nucleotide variations, including addition or deletion of one or more nucleotides or substitution with different nucleotides which polynucleotides retain the function or activity of the reference polynucleotide. In this regard, it is well understood in the art that certain alterations inclusive of mutations, additions, deletions and substitutions can be made to a reference polynucleotide whereby the altered polynucleotide retains the biological function or activity of the reference polynucleotide.

The term “polypeptide variant” refers to polypeptides in which one or more amino acids have been replaced by different amino acids and which retains the fuction or activity of the polypeptide. It is well understood in the art that some amino acids may be changed to others with broadly similar properties without changing the nature of the function or activity of the polypeptide (conservative substitutions) as described hereinafter.

By “primer” is meant an oligonucleotide which, when paired with a strand of DNA, is capable of initiating the synthesis of a primer extension product in the presence of a suitable polymerising agent. The primer is preferably single-stranded for maximum efficiency in amplification but can alternatively be double-stranded. A primer must be sufficiently long to prime the synthesis of extension products in the presence of the polymerisation agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotide residues, although it can contain fewer nucleotide residues. Primers can be large polynucleotides, such as from about 200 nucleotide residues to several kilobases or more. Primers can be selected to be “substantially complementary” to the sequence on the template to which it is designed to hybridise and serve as a site for the initiation of synthesis. By “substantially complementary”, it is meant that the primer is sufficiently complementary to hybridise with a target polynucleotide. Preferably, the primer contains no mismatches with the template to which it is designed to hybridise but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridise therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another nucleic acid, often called the “target nucleic acid”, through complementary base pairing. Probes may bind target nucleic acids lacking complete sequence complementarity with the probe, depending on the stringency of the hybridisation conditions. Probes can be labelled directly or indirectly.

The term “recombinant polynucleotide” or “synthetic polynucleotide” as used herein refers to a polynucleotide formed in vitro by the manipulation of nucleic acid into a form not normally found in nature. For example, the recombinant or synthetic polynucleotide may be in the form of an expression vector. Generally, such expression vectors include transcriptional and translational regulatory nucleic acid operably linked to the nucleotide sequence.

By “recombinant polypeptide” is meant a polypeptide made using recombinant techniques, i.e., through the expression of a recombinant or synthetic polynucleotide.

By “reporter molecule” as used in the present specification is meant a molecule that, by its chemical nature, provides an analytically identifiable signal, including for example, that allows the detection of a complex comprising an antigen-binding molecule and its target antigen. The term “reporter molecule” also extends to use of cell agglutination or inhibition of agglutination such as red blood cells on latex beads, and the like.

“Stringency” as used herein, refers to the temperature and ionic strength conditions, and presence or absence of certain organic solvents, during hybridisation. The higher the stringency, the higher will be the degree of complementarity between immobilised polynucleotides and the labelled polynucleotide.

“Stringent conditions” refers to temperature and ionic conditions under which only polynucleotides having a high frequency of complementary bases will hybridise. The stringency required is nucleotide sequence dependent and depends upon the various components present during hybridisation. Generally, stringent conditions are selected to be about 10 to 20° C. lower than the thermal melting point (Tₘ) for the specific sequence at a defined ionic strength and pH. The Tₘ is the temperature (under defined ionic strength and pH) at which 50% of a target sequence hybridises to a complementary probe.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software.

By “vector” is meant a polynucleotide molecule, preferably a DNA molecule derived, for example, from a plasmid, bacteriophage, or plant virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector can also include a selection marker such as an antibiotic resistance gene that can be used for selection of suitable transformants. Examples of such resistance genes are known to those of skill in the art and include the nptII gene that confers resistance to the antibiotics kanamycin and G418 (Geneticin®) and the hph gene which confers resistance to the antibiotic hygromycin B.

2. Recombinant Polypeptides, Polypeptide Fragments, Variants and Derivatives

2.1. Recombinant Polypeptides of the Invention

The invention provides a recombinant polypeptide comprising the sequence set forth in SEQ ID NO: 8, which corresponds to a mature polypeptide obtained from A. hydrophila, as described more fully hereinafter.

In one embodiment, the recombinant polypeptide may include a leader peptide comprising the sequence set forth in SEQ ID NO: 6 or biologically active fragment thereof, or variant or derivative of these which may be prepared using known methods and approaches as desribed infra. Accordingly, the invention also provides a recombinant precursor polypeptide according to SEQ ID NO: 2 or SEQ ID NO: 4, which comprises a leader peptide according to SEQ ID NO: 6 fused in frame with a polypeptide according to SEQ ID NO: 8.

2.2. Immuno-Interactive Fragments

Immuno-interactive fragments may be identified according to any suitable procedure known in the art. For example, a suitable method may include generating a fragment of a polypeptide according to any one of SEQ ID NO: 2, 4 or 8, administering the fragment to an animal, and detecting an immune response in the animal. Such response will include production of elements that protect said animal against infection by a bacterial species of Aeromonas, Vibrio or Edwardsiella or that specifically bind to the polypeptide according to any one of SEQ ID NO: 2, 4 or 8 or to said bacterial species.

Alternatively, an immuno-interactive fragment may be identified by combining a fragment of a polypeptide comprising a sequence set forth in any one of SEQ ID NO: 2, 4 and 8 with at least one antigen-binding molecule that specifically binds to said polypeptide, and detecting the presence of a conjugate comprising said fragment and said antigen-binding molecule. The conjugate may be detected using any suitable techniques as described in 2.3.1.

Prior to testing a particular fragment for immunoreactivity in the above method, a variety of predictive methods may be used to deduce whether a particular fragment can be used to obtain an antibody that cross-reacts with the native antigen. These predictive methods may be based on amino-terminal or carboxyl-terminal sequences as for example described in Chapter 11.14 of Ausubel et al., (1994-1998, supra). Alternatively, these predictive methods may be based on predictions of hydrophilicity as for example described by Kyte and Doolittle (1982, J. Mol. Biol. 157:105-132) and Hopp and Woods (1983, Mol. Immunol. 20:483489), or predictions of secondary structure as for example described by Choo and Fasman (1978, Ann. Rev. Biochem. 47:251-276).

Generally, peptide fragments consisting of 10 to 15 residues provide optimal results. Peptides as small as 6 or as large as 20 residues have worked successfully. Such peptide fragments may then be chemically coupled to a carrier molecule such as keyhole limpet hemocyanin (KLH) or bovine serum albumin (BSA) as for example described in Chapters 11.14 and 11.15 of Ausubel et al., (1994-1998, supra).

The peptides may be used to immunise a mammal as for example discussed in Example 1. Antibody titres against the native or parent polypeptide from which the peptide was selected may then be determined by radioimmunoassay or ELISA as for instance described in Chapters 11.16 and 114 of Ausubel et al., (1994-1998, supra), or by agglutination tests as described in Example 1.

Antibodies may then be purified from a suitable biological fluid of the animal by ammonium sulphate fractionation or by chromatography as is well known in the art. Exemplary protocols for antibody purification is given in Chapters 10.11 and 11.13 of Ausubel et al., (1994-1998, supra). Immunoreactivity of the antibody against the native or parent polypeptide may be determined by any suitable procedure such as, for example, western blot.

2.3. Polypeptide Variants

The invention also contemplates polypeptide variants of the recombinant polypeptide of the invention wherein said variants elicit an immune response, including the production of elements that protect said animal against infection by a bacterial species of a genus selected from the group consisting of Aeromonas, Vibrio and Edwardsiella or that specifically bind to the polypeptide according to any one of SEQ ID NO: 2, 4 or 8 or to said bacterial species.

Suitably, the polypeptide variants of the invention will cross-react with or mimic immunologically an epitope of the polypeptide according to any one of SEQ ID NO: 2, 4 or 8. Thus, polypeptide variants according to the invention may bind an antigen-binding molecule that also binds an epitope of the polypeptide according to any one of SEQ ID NO: 2, 4 or 8.

Suitable polypeptide variants may be identified by combining a compound suspected of being a variant with at least one antigen-binding molecule that binds to the said polypeptide. If a conjugate is formed comprising the compound and the antigen-binding molecule, this is indicative of the compound being a variant of a polypeptide of the invention.

In general, variants will be at least 75% homologous, more suitably at least 80%, preferably at least 85%, and more preferably at least 90% homologous to a polypeptide as for example shown in SEQ ID NO: 2, 4 or 8.

2.3.1. Assay Formats for Detecting Polypeptide Variants

Any suitable technique for determining formation of the conjugate may be used. For example, the antigen-binding molecule may be utilised in conventional immunoassays. Such immunoassays may include, but are not limited to, radioimmunoassays (RIAs), enzyme-linked immunosorbent assays (ELISAs) and immunochromatographic techniques (ICTs) which are well known those of skill in the art. For example, reference may be made to Coligan et al. (“CURRENT PROTOCOLS IN IMMUNOLOGY”, John Wiley & Sons, Inc, 1995-1997), in which a variety of immunoassays are described that may be used in accordance with the present invention. In this regard, the invention contemplates any immunoassay that can detect the presence of a conjugate as herein described. For example, immunoassays may include competitive and non-competitive assays as understood in the art. Such immunoassays may be carried out in solution or, at least in part, on solid supports, e.g., microtiter plates, polystyrene beads, nitrocellulose membranes, glass fibre membranes, immunochromatographic strips, and the like. The two most common formats for immunoassays are competitive and non-competitive (sandwich) formats.

In a competitive format, an antigen-binding molecule such as a polyclonal or monoclonal antibody is bound to a solid support. This antibody is suitably capable of binding a polypeptide according to any one of SEQ ID NO: 2, 4 or 8 or immuno-interactive fragment thereof A solution of antigen labelled to permit detection (e.g., a labelled polypeptide or immuno-interactive fragment) is allowed to compete with unlabelled antigen (e.g., a compound suspected of being a variant) for the solid phase antibody. The extent to which the labelled antigen is bound to the solid phase or is detected in the solution phase can be used as a measure of the presence of said conjugate.

In a non-competitive, or sandwich format, a polyclonal or preferably a monoclonal antibody is bound to a solid support. Such antibody is suitably capable of binding a polypeptide according to any one of SEQ ID NO: 2, 4 or 8 or immuno-interactive fragment thereof. In the case of a polyclonal antibody bound to the solid support, the sample containing the suspected antigen (i.e., a compound suspected of being said variant) is allowed to contact the solid phase in order for the antigen to bind to the antibody on the solid phase. Typically, after an incubation step, the sample is separated from the solid phase, which is then washed and incubated in the presence of additional polyclonal antibody that has been labelled to permit detection. Subsequently, the unbound labelled antibody is separated from the solid phase and the amount of labelled antibody in either the solution phase or bound to the solid phase in an antibody:antigen:antibody sandwich is determined as a measure of the presence of said conjugate. In the case of a non-competitive format employing monoclonal antibodies, a pair of monoclonal antibodies is typically utilised, one bound to the solid support and the other labelled to permit detection. The use of monoclonal antibody pairs that recognise different epitopic sites on an antigen makes it possible to conduct simultaneous immunometric assays in which the antigen and labelled antibody incubations do not require the intermediate steps of prior processes.

Alternatively, solid phase detection of the conjugate may be determined by immunoaffinity chromatography, as for example described by Coligan et al., (supra, in particular Chapter 9.5) and Ausubel et al. (“CURRENT PROTOCOLS IN MOLECULAR BIOLOGY”, John Wiley & Sons Inc, 1994-1998, in particular Chapter 10.11), by immunoblotting, as for example described by Ausubel et al. (supra, in Chapter 10.8), or by immunoprecipitation, as for example described by Ausubel et al. (supra, in Chapter 10.16).

Solution-phase immunoassays are also contemplated by the present invention. For instance, detection of said conjugate may be carried out in solution using flow cytometric analysis as for example described in Shapiro, H. M. (“PRACTICAL FLOW CYTOMETRY”, 3^{rd} ed., Wiley-Liss, New York, 1995).

2.3.2. Methods of Producing Polypeptide Variants

2.3.2.1. Mutagenesis

Polypeptide variants according to the invention can be identified either rationally, or via established methods of mutagenesis (see, for example, Watson, J. D. et al., “MOLECULAR BIOLOGY OF THE GENE”, Fourth Edition, Benjamin/Cummings, Menlo Park, Calif., 1987). Significantly, a random mutagenesis approach requires no a priori information about the gene sequence that is to be mutated. This approach has the advantage that it assesses the desirability of a particular mutant based on its function, and thus does not require an understanding of how or why the resultant mutant protein has adopted a particular conformation. Indeed, the random mutation of target gene sequences has been one approach used to obtain mutant proteins having desired characteristics (Leatherbarrow, R. 1986, J. Prot. Eng. 1: 7-16; Knowles, J. R., 1987, Science 236: 1252-1258; Shaw, W. V., 1987, Biochem. J. 246: 1-17; Gerit, J. A. 1987, Chem. Rev. 87: 1079-1105). Alternatively, where a particular sequence alteration is desired, methods of site-directed mutagenesis can be employed. Thus, such methods may be used to selectively alter only those amino acids of the protein that are believed to be important (Craik, C. S., 1985, Science 228: 291-297; Cronin, et al., 1988, Biochem. 27: 4572-4579; Wilks, et al., 1988, Science 242: 1541-1544).

Variant peptides or polypeptides, resulting from rational or established methods of mutagenesis or from combinatorial chemistries as hereinafter described, may comprise conservative amino acid substitutions. Exemplary conservative substitutions in an immuno-interactive polypeptide or polypeptide fragment according to the invention may be made according to the following table:

TABLE A

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile, |
| Phe | Met, Leu, Tyr |
| Ser | Thr |
| Thr | Ser |
| Trp | Tyr |
| Tyr | Trp, Phe |
| Val | Ile, Leu |

Substantial changes in function are made by selecting substitutions that are less conservative than those shown in TABLE A. Other replacements would be non-conservative substitutions and relatively fewer of these may be tolerated. Generally, the substitutions which are likely to produce the greatest changes in a polypeptide's properties are those in which (a) a hydrophilic residue (eg, Ser or Thr) is substituted for, or by, a hydrophobic residue (eg, Ala, Leu, Ile, Phe or Val); (b) a cysteinc or proline is substituted for, or by, any other residue; (c) a residue having an electropositive side chain (eg, Arg, His or Lys) is substituted for, or by, an electronegative residue (eg, Glu or Asp) or (d) a residue having a bulky side chain (eg, Phe or Trp) is substituted for, or by, one having a smaller side chain (eg, Ala, Ser) or no side chain (eg, Gly).

What constitutes suitable variants may be determined by conventional techniques. For example, nucleic acids encoding a polypeptide according to any one of SEQ ID NO: 2, 4 or 8 can be mutated using either random mutagenesis for example using transposon mutagenesis, or site-directed mutagenesis as described, for example, in Section 3.2 infra.

2.3.2.2. Peptide Libraries Produced by Combinatorial Chemistry

A number of facile combinatorial technologies can be utilised to synthesise molecular libraries of immense diversity. In the present case, variants of a polypeptide, or preferably a polypeptide fragment according to the invention, can be synthesised using such technologies. Variants can be screened subsequently using the methods described in Section 2.3.

Preferably, soluble synthetic peptide combinatorial libraries (SPCLs) are produced which offer the advantage of working with free peptides in solution, thus permitting adjustment of peptide concentration to accommodate a particular assay system. SPCLs are suitably prepared as hexamers. In this regard, a majority of binding sites is known to involve four to six residues. Cysteine is preferably excluded from the mixture positions to avoid the formation of disulfides and more difficult-to-define polymers. Exemplary methods of producing SPCLs are disclosed by Houghten et al. (1991, Nature 354: 84-86; 1992, BioTechniques 13: 412-421), Appel et al. (1992, Immunomethods 1: 17-23), and Pinilla et al. (1992, BioTechiniques 13: 901-905; 1993, Gene 128: 71-76).

Preparation of combinatorial synthetic peptide libraries may employ either t-butyloxycarbonyl (t-Boc) or 9-fluorenylmethyloxycarbonyl (Fmoc) chemistries (see Chapter 9.1, of Coligan et al., supra; Stewart and Young, 1984, Solid Phase Peptide Synthesis, 2nd ed. Pierce Chemical Co., Rockford, 111; and Atherton and Sheppard, 1989, Solid Phase Peptide Synthesis: A Practical Approach. IRL Press, Oxford) preferably, but not exclusively, using one of two different approaches. The first of these approaches, suitably termed the “split-process-recombine” or “split synthesis” method, was described first by Furka et al. (1988, 14th Int. Congr. Biochem., Prague, Czechoslovakia 5: 47; 1991, Int. J. Pept. Protein Res. 37: 487-493) and Lam et al. (1991, Nature 354: 82-84), and reviewed later by Eichler et al. (1995, Medicinal Research Reviews 15(6): 481-496) and Balkenhohl et al. (1996, Angew. Chem. Int. Ed. Engl. 35: 2288-2337). Briefly, the split synthesis method involves dividing a plurality of solid supports such as polymer beads into n equal fractions representative of the number of available amino acids for each step of the synthesis (e.g., 20 L-amino acids), coupling a single respective amino acid to each polymer bead of a corresponding fraction, and then thoroughly mixing the polymer beads of all the fractions together. This process is repeated for a total of x cycles to produce a stochastic collection of up to N^{x} different compounds. The peptide library so produced may be screened for example with a suitably labelled antigen-binding molecule that binds specifically to a polypeptide according to any one of SEQ ID NO: 2, 4 or 8. Upon detection, some of the positive beads are selected for sequencing to identify the active peptide. Such peptide may be subsequently cleaved from the beads, and assayed using the same antigen-binding molecule to identify the most active peptide sequence.

The second approach, the chemical ratio method, prepares mixed peptide resins using a specific ratio of amino acids empirically defined to give equimolar incorporation of each amino acid at each coupling step. Each resin bead contains a mixture of peptides. Approximate equimolar representation can be confirmed by amino acid analysis (Dooley and Houghten, 1993, Proc. Natl. Acad. Sci. U.S.A. 90: 10811-10815; Eichler and Houghten, 1993, Biochemistry 32: 11035-11041). Preferably, the synthetic peptide library is produced on polyethylene rods, or pins, as a solid support, as for example disclosed by Geysen et al. (1986, Mol. Immunol. 23: 709-715). An exemplary peptide library of this type may consist of octapeptides in which the third and fourth position are defined with each of the 20 amino acids, whereas the remaining six positions are present as mixtures. This peptide library can be represented by the formula Ac-XXO₁O₂XXXX-S₅, where S₅ is the solid support. Peptide mixtures remain on the pins when assayed against a soluble receptor molecule. For example, the peptide library of Geysen (1986, Immun. Today 6: 364-369; and Geysen et al., Ibid), comprising for example dipeptides, is first screened for the ability to bind to a target molecule. The most active dipeptides are then selected for an additional round of testing comprising linking, to the starting dipeptide, an additional residue (or by internally modifying the components of the original starting dipeptide) and then screening this set of candidates for the desired activity. This process is reiterated until the binding partner having the desired properties is identified.

2.3.2.3. Alanine Scanning Mutagenesis

In one embodiment, the invention herein utilises a systematic analysis of a polypeptide or polypeptide fragment according to the invention to determine the residues in the polypeptide or fragment that are involved in the interaction with a hyaluronidase substrate (i.e., hyaluronic acid). Such analysis is conveniently performed using recombinant DNA technology. In general, a DNA sequence encoding the polypeptide or fragment is cloned and manipulated so that it may be expressed in a convenient host. DNA encoding the polypeptide or fragment can be obtained from a genomic library, from cDNA derived from mRNA in cells expressing the said polypeptide or fragment, or by synthetically constructing the DNA sequence (Sambrook et al., supra; Ausubel et al., supra).

The wild-type DNA encoding the polypeptide or fragment is then inserted into an appropriate plasmid or vector as described herein. In particular, prokaryotes are preferred for cloning and expressing DNA sequences to produce variants of the polypeptide or fragment. For example, E. coli K12 strain 294 (ATCC No. 31446) may be used, as well as E. coli B, E. coli X1776 (ATCC No. 31537), and E. coli c600 and c600hfl, and E. coli W3110 (F⁻, ⁻, prototrophic, ATCC No. 27325), bacilli such as Bacillus subtilis, and other Enterobacteriaceae such as Salmonella typhimurium or Serratia marcescens, and various Pseudomonas species. A preferred prokaryote is E. coli W3110 (ATCC 27325).

Once the polypeptide or fragment is cloned, site-specific mutagenesis as for example described by Carter et al. (1986, Nucl. Acids. Res., 13: 4331) or by Zoller et al. (1987, Nucl. Acids Res., 10: 6487), cassette mutagenesis as for example described by Wells et al. (1985, Gene, 34: 315), restriction selection mutagenesis as for example described by Wells et al. (1986, Philos. Trans. R. Soc. London SerA, 317: 415), or other known techniques may be performed on the cloned DNA to produce the variant DNA that encodes for the changes in amino acid sequence defined by the residues being substituted. When operably linked to an appropriate expression vector, variants are obtained. In some cases, recovery of the variant may be facilitated by expressing and secreting such molecules from the expression host by use of an appropriate signal sequence operably linked to the DNA sequence encoding the variant. Such methods are well known to those skilled in the art. Of course, other methods may be employed to produce such polypeptides or fragments such as the in vitro chemical synthesis of the desired polypeptide variant (Barany et al. In The Peptides, eds. E. Gross and J. Meienhofer (Academic Press: N.Y. 1979), Vol. 2, pp. 3-254).

Once the different variants are produced, they are contacted with an antigen-binding molecule that binds a polypeptide according to any one of SEQ ID NO: 2, 4 or 8 or immuno-interactive fragment thereof and the interaction, if any, between the antigen-binding molecule and each variant is determined. These activities are compared to the activity of the wild-type polypeptide or immuno-interactive fragment with the same antigen-binding molecule to determine which of the amino acid residues in the active domain or epitope are involved in the interaction with the antigen-binding molecule. The scanning amino acid used in such an analysis may be any different amino acid from that substituted, i.e., any of the 19 other naturally occurring amino acids.

The interaction between the antigen-binding molecule and parent and variant can be measured by any convenient assay as for example described herein. While any number of analytical measurements may be used to compare activities, a convenient one for binding of antigen-binding molecule is the dissociation constant K_{d} of the complex formed between the variant and antigen-binding molecule as compared to the K_{d} for the wild-type immuno-interactive fragment. Generally, a two-fold increase or decrease in K_{d} per analogous residue substituted by the substitution indicates that the substituted residue(s) is active in the interaction of the wild-type polypeptide or immuno-interactive fragment with the target antigen-binding molecule.

When a suspected or known active amino acid residue is subjected to scanning amino acid analysis, the amino acid residues immediately adjacent thereto should be scanned. Three residue-substituted polypeptides can be made. One contains a scanning amino acid, preferably alanine, at position N that is the suspected or known active amino acid. The two others contain the scanning amino acid at position N+1 and N−1. If each substituted polypeptide or fragment causes a greater than about two-fold effect on the rate of cumulus dispersal of an oocyte-cumulus complex, the scanning amino acid is substituted at position N+2 and N—2. This is repeated until at least one, and preferably four, residues are identified in each direction which have less than about a two-fold effect on the said rate or either of the ends of the parent polypeptide or fragment are reached. In this manner, one or more amino acids along a continuous amino acid sequence that are involved in the interaction with the particular antigen-binding molecule can be identified.

The active amino acid residue identified by amino acid scan is typically one that contacts the receptor target (antigen-binding molecule) directly. However, active amino acids may also indirectly contact the target through salt bridges formed with other residues or small molecules such as H₂O or ionic species such as Na⁺, Ca⁺², Mg⁺², or Zn^{+2.}

In some cases, the substitution of a scanning amino acid at one or more residues results in a residue-substituted polypeptide which is not expressed at levels that allow for the isolation of quantities sufficient to carry out analysis of its activity with the receptor. In such cases, a different scanning amino acid, preferably an isosteric amino acid, can be used.

Among the preferred scanning amino acids are relatively small, neutral amino acids. Such amino acids include alanine, glycine, serine, and cysteine. Alanine is the preferred scanning amino acid among this group because it eliminates the side-chain beyond the beta-carbon and is less likely to alter the main-chain conformation of the variant. Alanine is also preferred because it is the most common amino acid. Further, it is frequently found in both buried and exposed positions (Creighton, The Proteins, W. H. Freeman & Co., N.Y.; Chothia, 1976, J. Mol. Biol., 150: 1). If alanine substitution does not yield adequate amounts of variant, an isosteric amino acid can be used. Alternatively, the following amino acids in decreasing order of preference may be used: Ser, Asn, and Leu.

Once the active amino acid residues are identified, isosteric amino acids may be substituted. Such isosteric substitutions need not occur in all instances and may be performed before any active amino acid is identified. Such isosteric amino acid substitution is performed to minimise the potential disruptive effects on conformation that some substitutions can cause. Isosteric amino acids are shown in the table below:

TABLE B

| Polypeptide Amino Acid | Isosteric Scanning Amino Acid |
|---|---|
| Ala (A) | Ser, Gly |
| Glu (E) | Gln, Asp |
| Gln (Q) | Asn, Glu |
| Asp (D) | Asn, Glu |
| Asn (N) | Ala, Asp |
| Leu (L) | Met, Ile |
| Gly (G) | Pro, Ala |
| Lys (K) | Met, Arg |
| Ser (S) | Thr, Ala |
| Val (V) | Ile, Thr |
| Arg (R) | Lys, Met, Asn |
| Thr (T) | Ser, Val |
| Pro (P) | Gly |
| Ile (I) | Met, Leu, Val |
| Met (M) | Ile, Leu |
| Phe (F) | Tyr |
| Tyr (Y) | Phe |
| Cys (C) | Ser, Ala |
| Trp (W) | Phe |
| His (H) | Asn, Gln |

The method herein can be used to detect active amino acid residues within different epitopes of a polypeptide or immuno-interactive fragment according to the invention. Once this identification is made, various modifications to the wild-type polypeptide or immuno-interactive fragment may be made to modify the interaction between the parent polypeptide/immuno-interactive fragment and one or more of the targets.

2.3.2.4. Polypeptide or Peptide Libraries Produced by Phage Display

The identification of variants can also be facilitated through the use of a phage (or phagemid) display protein ligand screening system as for example described by Lowman, et al. (1991, Biochem. 30:10832-10838), Markland, et al. (1991, Gene 109:13-19), Roberts, et al. (1992, Proc. Natl. Acad. Sci. (U.S.A.) 89:2429-2433), Smith, G. P. (1985, Science 228:1315-1317), Smith, et al. (1990, Science 248:1126-1128) and Lardner et al. (U.S. Pat. No. 5,223,409). In general, this method involves expressing a fusion protein in which the desired protein ligand is filsed to the N-terminus of a viral coat protein (such as the M13 Gene III coat protein, or a lambda coat protein).

In one embodiment, a library of phage is engineered to display novel peptides within the phage coat protein sequences. Novel peptide sequences are generated by random mutagenesis of gene fragments encoding an immuno-interactive polypeptide fragment using error-prone PCR, or by in vivo mutation by E. coli mutator cells. The novel peptides displayed on the surface of the phage are placed in contact, with an antigen binding molecule such as an antibody or antibody fragment against the particular immuno-interactive fragment on which the novel peptide sequences are based. Phage that display coat protein having peptides that are capable of binding to such antibodies are immobilised by such treatment, whereas all other phage can be washed away. After the removal of unbound phage, the bound phage can be amplified, and the DNA encoding their coat proteins can be sequenced. In this manner, the amino acid sequence of the embedded peptide or polypeptide can be deduced.

In more detail, the method involves (a) constructing a replicable expression vector comprising a first gene encoding a polypeptide or immuno-interactive fragment of the invention, a second gene encoding at least a portion of a natural or wild-type phage coat protein wherein the first and second genes are heterologous, and a transcription regulatory element operably linked to the first and second genes, thereby forming a gene fusion encoding a fusion protein; (b) mutating the vector at one or more selected positions within the first gene thereby forming a family of related plasmids; (c) transforming suitable host cells with the plasmids; (d) infecting the transformed host cells with a helper phage having a gene encoding the phage coat protein; (e) culturing the transformed infected host cells under conditions suitable for forming recombinant phagemid particles containing at least a portion of the plasmid and capable of transforming the host, the conditions adjusted so that no more than a minor amount of phagemid particles display more than one copy of the fusion protein on the surface of the particle; (f) contacting the phagemid particles with an antigen-binding molecule that binds to the polypeptide or immuno-interactive fragment so that at least a portion of the phagemid particles bind to the antigen-binding molecule; and (g) separating the phagemid particles that bind from those that do not. Preferably, the method further comprises transforming suitable host cells with recombinant phagemid particles that bind to the antigen-binding molecule and repeating steps (d) through (g) one or more times.

Preferably in this method the plasmid is under tight control of the transcription regulatory element, and the culturing conditions are adjusted so that the amount or number of phagemid particles displaying more than one copy of the fusion protein on the surface of the particle is less than about 1%. Also, preferably, the amount of phagemid particles displaying more than one copy of the fusion protein is less than 10% of the amount of phagemid particles displaying a single copy of the fusion protein. Most preferably, the amount is less than 20%.

Typically in this method, the expression vector will further contain a secretory signal sequence fused to the DNA encoding each subunit of the polypeptide and the transcription regulatory element will be a promoter system. Preferred promoter systems are selected from lac Z, λ_{PL}, tac, T7 polymerase, tryptophan, and alkaline phosphatase promoters and combinations thereof. Also, normally the method will employ a helper phage selected from M13K07, M13R408, M13-VCS, and Phi X 174. The preferred helper phage is M13K07, and the preferred coat protein is the M13 Phage gene III coat protein. The preferred host is E. coli, and protease-deficient strains of E. coli.

Repeated cycles of variant selection are used to select for higher and higher affinity binding by the phagemid selection of multiple amino acid changes that are selected by multiple selection cycles. Following a first round of phagemid selection, involving a first region or selection of amino acids in the ligand polypeptide, additional rounds of phagemid selection in other regions or amino acids of the ligand polypeptide are conducted. The cycles of phagemid selection are repeated until the desired affinity properties of the ligand polypeptide are achieved.

It will be appreciated that the amino acid residues that form the binding domain of the polypeptide or immuno-interactive fragment may not be sequentially linked and may reside on different subunits of the polypeptide. That is, the binding domain tracks with the particular secondary structure at the binding site and not the primary structure. Thus, generally, mutations will be introduced into codons encoding amino acids within a particular secondary structure at sites directed away from the interior of the polypeptide so that they will have the potential to interact with the antigen-binding molecule.

The phagemid-display method herein contemplates fusing a polynucleotide encoding the polypeptide or immuno-interactive fragment (polynucleotide 1) to a second polynucleotide (polynucleotide 2) such that a fusion protein is generated during transcription. Polynucleotide 2 is typically a coat protein gene of a phage, and preferably it is the phage M13 gene III coat protein, or a fragment thereof. Fusion of polynucleotides 1 and 2 may be accomplished by inserting polynucleotide 2 into a particular site on a plasmid that contains polynucleotide 1, or by inserting polynucleotide 1 into a particular site on a plasmid that contains polynucleotide 2.

Between polynucleotide 1 and polynucleotide 2, DNA encoding a termination codon may be inserted, such termination codons being UAG (amber), UAA (ocher), and UGA (opel) (see for example, Davis et al., Microbiology (Harper and Row: New York, 1980), pages 237, 245-247, and 274). The termination codon expressed in a wild-type host cell results in the synthesis of the polynucleotide 1 protein product without the polynucleotide 2 protein attached. However, growth in a suppressor host cell results in the synthesis of detectable quantities of fused protein. Such suppressor host cells contain a tRNA modified to insert an amino acid in the termination codon position of the mRNA, thereby resulting in production of detectable amounts of the fusion protein. Such suppressor host cells are well known and described, such as E. coli suppressor strain (Bullock et al., 1987, BioTechniques, 5: 376-379). Any acceptable method may be used to place such a termination codon into the mRNA encoding the fusion polypeptide.

The suppressible codon may be inserted between the polynucleotide encoding the immuno-interactive fragment and a second polynucleotide encoding at least a portion of a phage coat protein. Alternatively, the suppressible termination codon may be inserted adjacent to the fusion site by replacing the last amino acid triplet in the polypeptide or the first amino acid in the phage coat protein. When the phagemid containing the suppressible codon is grown in a suppressor host cell, it results in the detectable production of a fusion polypeptide containing the polypeptide or immuno-interactive fragment and the coat protein. When the phagemid is grown in a non-suppressor host cell, the polypeptide or immuno-interactive fragment is synthesised substantially without fusion to the phage coat protein due to termination at the inserted suppressible triplet encoding UAG, UAA, or UGA. In the non-suppressor cell the polypeptide is synthesised and secreted from the host cell due to the absence of the fused phage coat protein which otherwise anchored it to the host cell.

The polypeptide or immuno-interactive fragment may be altered at one or more selected codons. An alteration is defined as a substitution, deletion, or insertion of one or more codons in the gene encoding the polypeptide or immuno-interactive fragment that results in a change in the amino acid sequence of the polypeptide or immuno-interactive fragment as compared with the unaltered or native sequence of the said polypeptide or fragment. Preferably, the alterations will be by substitution of at least one amino acid with any other amino acid in one or more regions of the molecule. The alterations may be produced by a variety of methods known in the art. These methods include, but are not limited to, oligonucleotide-mediated mutagenesis and cassette mutagenesis as described fro example herein.

For preparing the antigen-binding molecule and binding it with the phagemid, the antigen-binding molecule is attached to a suitable matrix such as agarose beads, acrylamide beads, glass beads, cellulose, various acrylic copolymers, hydroxyalkyl methacrylate gels, polyacrylic acid, polymethacrylic copolymers, nylon, neutral and ionic carriers, and the like. Attachment of the antigen-binding molecule to the matrix may be accomplished by methods described in Methods Enzymol., 44: (1976), or by other means known in the art.

After attachment of the antigen-binding molecule to the matrix, the immobilised target is contacted with the library of phagemid particles under conditions suitable for binding of at least a portion of the phagemid particles with the immobilised target. Normally, the conditions, including pH, ionic strength, temperature, and the like will mimic physiological conditions.

Bound phagemid particles (“binders”) having high affinity for the immobilised receptor are separated from those having a low affinity (and thus do not bind to the target) by washing. Binders may be dissociated from the immobilised target by a variety of methods. These methods include competitive dissociation using the wild-type ligand, altering pH and/or ionic strength, and methods known in the art.

Suitable host cells are infected with the binders and helper phage, and the host cells are cultured under conditions suitable for amplification of the phagemid particles. The phagemid particles are then collected and the selection process is repeated one or more times until binders having the desired affinity for the target molecule are selected.

2.3.2.5. Rational Drug Design

Variants of naturally occurring polypeptides or immuno-interactive fragments according to the invention may also be obtained using the principles of conventional or of rational drug design as for example described by Andrews, et al. (In: “PROCEEDINGS OF THE ALFRED BENZON SYMPOSIUM”, volume 28, pp. 145-165, Munksgaard, Copenhagen, 1990), McPherson, A. (1990, Eur. J. Biochem. 189:1-24), Hol, et al. (In: “MOLECULAR RECOGNITION: CHEMICAL AND BIOCHEMICAL PROBLEMS”, Roberts, S. M. (ed.); Royal Society of Chemistry; pp. 84-93, 1989), Hol, W. G. J. (1989, Arzneim-Forsch. 39:1016-1018), Hol, W. G. J. (1986, Agnew Chem. Int. Ed. Engl. 25:767-778).

In accordance with the methods of conventional drug design, the desired variant molecules are obtained by randomly testing molecules whose structures have an attribute in common with the structure of a “native” or “wild-type” polypeptide or immuno-interactive fragment according to the invention. The quantitative contribution that results from a change in a particular group of a binding molecule can be determined by measuring the capacity of competition or cooperativity between the native polypeptide or immuno-interactive fragment and the putative polypeptide variant.

In one embodiment of rational drug design, the polypeptide variant is designed to share an attribute of the most stable three-dimensional conformation of a polypeptide or immuno-interactive fragment according to the invention. Thus, the variant may be designed to possess chemical groups that are oriented in a way sufficient to cause ionic, hydrophobic, or van der Waals interactions that are similar to those exhibited by the polypeptide or immuno-interactive fragment. In a second method of rational design, the capacity of a particular polypeptide or immuno-interactive fragment to undergo conformational “breathing” is exploited. Such “breathing”—the transient and reversible assumption of a different molecular conformation—is a well-appreciated phenomenon, and results from temperature, thermodynamic factors, and from the catalytic activity of the molecule. Knowledge of the 3-dimensional structure of the polypeptide or immuno-interactive fragment facilitates such an evaluation. An evaluation of the natural conformational changes of an polypeptide or immuno-interactive fragment facilitates the recognition of potential hinge sites, potential sites at which hydrogen bonding, ionic bonds or van der Waals bonds might form or might be eliminated due to the breathing of the molecule, etc. Such recognition permits the identification of the additional conformations that the polypeptide or immuno-interactive fragment could assume, and enables the rational design and production of immunomimetics that share such conformations.

The preferred method for performing rational immunomimetic design employs a computer system capable of forming a representation of the three-dimensional structure of the polypeptide or immuno-interactive fragment (such as those obtained using RIBBON (Priestle, J., 1988, J. Mol. Graphics 21:572), QUANTA (Polygen), InSite (Biosyn), or Nanovision (American Chemical Society)). Such analyses are exemplified by Hol, et al. (In: “MOLECULAR RECOGNITION: CHEMICAL AND BIOCHEMICAL PROBLEMS”, supra, Hol, W. G. J. (1989, supra) and Hol, W. G. J., (1986, supra).

In lieu of such direct comparative evaluations of putative polypeptide variants, screening assays may be used to identify such molecules. Such assays will preferably exploit the capacity of the variant to bind to an antigen-binding molecule as described in Section 2.3.1.

2.4. Polypeptide Derivatives

With reference to suitable derivatives of the invention, such derivatives include amino acid deletions and/or additions to the polypeptide, fragment or variant of the invention, wherein said derivatives elicit an immune response in an animal, including elements that specifically bind to the polypeptide, fragment or variant. “Additions” of amino acids may include fusion of the polypeptide, fragment or variant of the invention with other polypeptides or proteins. For example, it will be appreciated that said polypeptide, fragment or variant may be incorporated into larger polypeptides, and that such larger polypeptides may also be expected to elicit the said immune response.

A polypeptide, fragment or variant according to the invention may be fused to a further protein, for example, which is not derived from the original host. The further protein may assist in the purification of the fusion protein. For instance, a polyhistidine tag or a maltose binding protein may be used in this respect as described in more detail below.

Other possible fusion proteins are those which produce an immunomodulatory response. Particular examples of such proteins include Protein A or glutathione S-transferase (GST).

Other derivatives contemplated by the invention include, but are not limited to, modification to side chains, incorporation of unnatural amino acids and/or their derivatives during peptide, polypeptide or protein synthesis and the use of crosslinkers and other methods which impose conformational constraints on the polypeptide, fragment or variant of the invention.

Examples of side chain modifications contemplated by the present invention include modifications of amino groups such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with NaBH₄; reductive alkylation by reaction with an aldehyde followed by reduction with NaBH₄; and trinitrobenzylation of amino groups with 2, 4, 6-trinitrobenzene sulphonic acid (TNBS).

The carboxyl group may be modified by carbodiimide activation via O-acylisourea formation followed by subsequent derivitisation, by way of example, to a corresponding amide.

The guanidine group of arginine residues may be modified by formation of heterocyclic condensation products with reagents such as 2,3-butanedione, phenylglyoxal and glyoxal.

Sulphydryl groups may be modified by methods such as performic acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulphides with other thiol compounds; reaction with maleimide, maleic anhydride or other substituted maleimide; carboxymethylation with iodoacetic acid or iodoacetamide; and carbamoylation with cyanate at alkaline pH.

Tryptophan residues may be modified, for example, by alkylation of the indole ring with 2-hydroxy-5-nitrobenzyl bromide or sulphonyl halides or by oxidation with N-bromosuccinimide.

Tyrosine residues may be modified by nitration with tetranitromethane to form a 3-nitrotyrosine derivative.

The imidazole ring of a histidine residue may be modified by N-carbethoxylation with diethylpyrocarbonate or by alkylation with iodoacetic acid derivatives.

Examples of incorporating unnatural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, omithine, sarcosine, 2-thienyl alanine and/or D-isomers of amino acids. A list of unnatural amino acids contemplated by the present invention is shown in TABLE C.

TABLE C

| | NON-CONVENTIONAL |
|---|---|
| Non-conventional amino acid | AMINO ACID |
| -aminobutyric acid | L-N-methylalanine |
| -amino- -methylbutyrate | L-N-methylarginine |
| aminocyclopropane-carboxylate | L-N-methylasparagine |
| aminoisobutyric acid | L-N-methylaspartic acid |
| aminonorbornyl-carboxylate | L-N-methylcysteine |
| cyclohexylalanine | L-N-methylglutamine |
| cyclopentylalanine | L-N-methylglutamic acid |
| L-N-methylisoleucine | L-N-methylhistidine |
| D-alanine | L-N-methylleucine |
| D-arginine | L-N-methyllysine |
| D-aspartic acid | L-N-methylmethionine |
| D-cysteine | L-N-methylnorleucine |
| D-glutamate | L-N-methylnorvaline |
| D-glutamic acid | L-N-methylornithine |
| D-histidine | L-N-methylphenylalanine |
| D-isoleucine | L-N-methylproline |
| D-leucine | L-N-medlylserine |
| D-lysine | L-N-methylthreonine |
| D-methionine | L-N-methyltryptophan |
| D-ornithine | L-N-methyltyrosine |
| D-phenylalanine | L-N-methylvaline |
| D-proline | L-N-methylethylglycine |
| D-serine | L-N-methyl-t-butylglycine |
| D-threonine | L-norleucine |
| D-tryptophan | L-norvaline |
| D-tyrosine | -methyl-aminoisobutyrate |
| D-valine | -methyl- -aminobutyrate |
| D- -methylalanine | -methylcyclohexylalanine |
| D- -methylarginine | -methylcylcopentylalanine |
| D- -methylasparagine | -methyl- -napthylalanine |
| D- -methylaspartate | -methylpenicillamine |
| D- -methylcysteine | N-(4-aminobutyl)glycine |
| D- -methylglutamine | N-(2-aminoethyl)glycine |
| D- -methylhistidine | N-(3-aminopropyl)glycine |
| D- -methylisoleucine | N-amino- -methylbutyrate |
| D- -methylleucine | -napthylalanine |
| D- -methyllysine | N-benzylglycine |
| D- -methylmethionine | N-(2-carbamylediyl)glycine |
| D- -methylornithiine | N-(carbamylmethyl)glycine |
| D- -methylphenylalanine | N-(2-carboxyethyl)glycine |
| D- -methylproline | N-(carboxymethyl)glycine |
| D- -methylserine | N-cyclobutylglycine |
| D- -methylthreonine | N-cycloheptylglycine |
| D- -methyltryptophan | N-cyclohexylglycine |
| D- -methyltyrosine | N-cyclodecylglycine |
| L- -methylleucine | L- -methyllysine |
| L- -methylmethionine | L- -methylnorleucine |
| L- -methylnorvatine | L- -methylornithine |
| L- -methylphenylalanine | L- -methylproline |
| L- -methylserine | L- -methylthreonine |
| L- -methyltryptophan | L- -methyltyrosine |
| L- -methylvaline | L-N-methylhomophenylalanine |
| N-(N-(2,2-diphenylethyl | N-(N-(3,3-diphenylpropyl |
| carbamylmethyl)glycine | carbamylmethyl)glycine |
| 1-carboxy-1-(2,2-diphenyl-ethyl | |
| amino)cyclopropane | |

Also contemplated is the use of crosslinkers, for example, to stabilise 3D conformations of the polypeptides, fragments or variants of the invention, using homo-bifunctional cross linkers such as bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety or carbodiimide. In addition, peptides can be conformationally constrained, for example, by introduction of double bonds between C and C atoms of amino acids, by incorporation of C and N-methylamino acids, and by formation of cyclic peptides or analogues by introducing covalent bonds such as forming an amide bond between the N and C termini between two side chains or between a side chain and the N or C terminus of the peptides or analogues. For example, reference may be made to: Marlowe (1993, Biorganic & Medicinal Chemistry Letters 3: 437-44) who describes peptide cyclisation on TFA resin using trimethylsilyl (TMSE) ester as an orthogonal protecting group; Pallin and Tan (1995, J. Chem. Soc. Chem. Comm. 2021-2022) who describe the cyclisation of unprotected peptides in aqueous solution by oxime formation; Algin et al (1994, Tetrahedron Letters 35: 9633-9636) who disclose solid-phase synthesis of head-to-tail cyclic peptides via lysine side-chain anchoring; Kates et al (1993, Tetrahedron Letters 34: 1549-1552) who describe the production of head-to-tail cyclic peptides by three-dimensional solid phase strategy; Tumelty et al (1994, J. Chem. Soc. Chem. Comm. 1067-1068) who describe the synthesis of cyclic peptides from an immobilised activated intermediate, wherein activation of the immobilised peptide is carried out with N-protecting group intact and subsequent removal leading to cyclisation; McMurray et al (1994, Peptide Research 7: 195-206) who disclose head-to-tail cyclisation of peptides attached to insoluble supports by means of the side chains of aspartic and glutamic acid; Hruby et al (1994, Reactive Polymers 22: 231-241) who teach an alternate method for cyclising peptides via solid supports; and Schmidt and Langer (1997, J Peptide Res. 49: 67-73) who disclose a method for synthesising cyclotetrapeptides and cyclopentapeptides. The foregoing methods may be used to produce conformationally constrained polypeptides that have hyaluronidase activity and activity for dispersing cumulus cells from an oocyte-cumulus complex.

The invention also contemplates polypeptides, fragments or variants of the invention that have been modified using ordinary molecular biological techniques so as to improve their resistance to proteolytic degradation or to optimise solubility properties or to render them more suitable as an immunogenic agent.

2.5. Methods of Preparing the Polypeptides of the Invention

Polypeptides of the inventions may be prepared by any suitable procedure known to those of skill in the art. For example, the polypeptides may be prepared by a procedure including the steps of:—

(a) preparing a recombinant polynucleotide comprising a nucleotide sequence encoding a polypeptide comprising the sequence set forth in SEQ ID NO: 2, 4 or 8, or variant or derivative of these, which nucleotide sequence is operably linked to transcriptional and translational regulatory nucleic acid;

(b) introducing the recombinant polynucleotide into a suitable host cell;

(c) culturing the host cell to express recombinant polypeptide from said recombinant polynucleotide; and

(d) isolating the recombinant polypeptide.

Suitably, said nucleotide sequence comprises the sequence set forth in any one of SEQ ID NO: 1, 3 or 7.

The recombinant polynucleotide preferably comprises either an expression vector that may be a self-replicating extra-chromosomal vector such as a plasmid, or a vector that integrates into a host genome.

The transcriptional and translational regulatory nucleic acid will generally be appropriate for the host cell used for expression. Numerous types of appropriate expression vectors and suitable regulatory sequences are known in the art for a variety of host cells.

Typically, the transcriptional and translational regulatory nucleic acid may include, but is not limited to, promoter sequences, leader or signal sequences, ribosomal binding sites, transcriptional start and stop sequences, translational start and termination sequences, and enhancer or activator sequences.

Constitutive or inducible promoters as known in the art are contemplated by the invention. The promoters may be either naturally occurring promoters, or hybrid promoters that combine elements of more than one promoter.

In a preferred embodiment, the expression vector contains a selectable marker gene to allow the selection of transformed host cells. Selection genes are well known in the art and will vary with the host cell used.

The expression vector may also include a fusion partner (typically provided by the expression vector) so that the recombinant polypeptide of the invention is expressed as a fusion polypeptide with said fusion partner. The main advantage of fusion partners is that they assist identification and/or purification of said fusion polypeptide.

In order to express said fusion polypeptide, it is necessary to ligate a polynucleotide according to the invention into the expression vector so that the translational reading frames of the fusion partner and the polynucleotide coincide.

Well known examples of fusion partners include, but are not limited to, glutathione-S-transferase (GST), Fc potion of human IgG, maltose binding protein (MBP) and hexahistidine (HIS₆), which are particularly useful for isolation of the fusion polypeptide by affinity chromatography. For the purposes of fusion polypeptide purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system. In a preferred embodiment, the recombinant polynucleotide is expressed in the commercial vector QIAexpress™ pQE-30 as described more fully hereinafter.

Another fusion partner well known in the art is green fluorescent protein (GFP). This fusion partner serves as a fluorescent “tag” which allows the fusion polypeptide of the invention to be identified by fluorescence microscopy or by flow cytometry. The GFP tag is useful when assessing subcellular localisation of the fusion polypeptide of the invention, or for isolating cells which express the fusion polypeptide of the invention. Flow cytometric methods such as fluorescence activated cell sorting (FACS) are particularly useful in this latter application.

Preferably, the fusion partners also have protease cleavage sites, such as for Factor Xₐ or Thrombin, which allow the relevant protease to partially digest the fusion polypeptide of the invention and thereby liberate the recombinant polypeptide of the invention therefrom. The liberated polypeptide can then be isolated from the fusion partner by subsequent chromatographic separation.

Fusion partners according to the invention also include within their scope “epitope tags”, which are usually short peptide sequences for which a specific antibody is available. Well known examples of epitope tags for which specific monoclonal antibodies are readily available include c-Myc, influenza virus, haemagglutinin and FLAG tags.

The step of introducing into the host cell the recombinant polynucleotide may be effected by any suitable method including transfection, and transformation, the choice of which will be dependent on the host cell employed. Such methods are well known to those of skill in the art.

Recombinant polypeptides of the invention may be produced by culturing a host cell transformed with an expression vector containing nucleic acid encoding a polypeptide, fragment, variant or derivative according to the invention. The conditions appropriate for protein expression will vary with the choice of expression vector and the host cell. This is easily ascertained by one skilled in the art through routine experimentation.

Suitable host cells for expression may be prokaryotic or eukaryotic. One preferred host cell for expression of a polypeptide according to the invention is a bacterium. The bacterium used may be Escherichia coli. Alternatively, the host cell may be an insect cell such as, for example, SF9 cells that may be utilised with a baculovirus expression system.

The recombinant protein may be conveniently prepared by a person skilled in the art using standard protocols as for example described in Sambrook, et al., MOLECULAR CLONING. A LABORATORY MANUAL (Cold Spring Harbor Press, 1989), in particular Sections 16 and 17; Ausubel et al., CURRENT PROTOCOLS IN MOLECULAR BIOLOGY (John Wiley & Sons, Inc. 1994-1998), in particular Chapters 10 and 16; and Coligan et al., CURRENT PROTOCOLS IN PROTEIN SCIENCE (John Wiley & Sons, Inc. 1995-1997), in particular Chapters 1, 5 and 6.

Alternatively, the polypeptide, fragments, variants or derivatives of the invention may be synthesised using solution synthesis or solid phase synthesis as described, for example, in Chapter 9 of Atherton and Shephard (supra) and in Roberge et al (1995, Science 269: 202).

3. Polynucleotides of the Invention

3.1. Polynucleotides Encoding Polypeptides of the Invention

The invention further provides a polynucleotide that encodes a polypeptide, fragment, variant or derivative as defined above. Suitably, the polynucleotide comprises the entire sequence of nucleotides set forth in SEQ ID NO: 1. SEQ ID NO: 1 corresponds to an 1810 bp DNA sequence obtained by PCR amplification as will be more fully described hereinafter. This sequence defines: (1) a 5′ untranslated region from nucleotide 1 through nucleotide 480 of SEQ ID NO: 1; (2) an open reading frame from nucleotide 481 through nucleotide 1602; and (3) a 3′ untranslated region from nucleotide 1603 through nucleotide 1810. The aforementioned open reading frame encodes a precursor polypeptide comprising a leader peptide encoded by nucleotides 481 through 540, and a mature polypeptide encoded by nucleotides 541 through 1602. Suitably, the polynucleotide comprises the sequence set forth in SEQ ID NO: 3. SEQ ID NO: 3 defines the aforementioned open reading frame and thus encodes the said precursor polypeptide. Preferably, the polynucleotide comprises the sequence set forth in SEQ ID NO: 7, which corresponds to nucleotide 541 through nucleotide 1602 and thus encodes the said mature polypeptide. SEQ ID NO: 5 corresponds to nucleotide 481 through 540 of SEQ ID NO: 1 and thus encodes the leader polypeptide of the aforementioned precursor polypeptide.

3.2. Polynucleotides Variants

In general, polynucleotide variants according to the invention comprise regions that show at least 70%, more suitably at least 80%, preferably at least 90%, and most preferably at least 95% sequence identity over a reference polynucleotide sequence of identical size (“comparison window”) or when compared to an aligned sequence in which the alignment is performed by a computer homology program known in the art. What constitutes suitable variants may be determined by conventional techniques. For example, a polynucleotide according to any one of SEQ ID NO: 1, 3, 5 or 7 can be mutated using random mutagenesis (e.g., transposon mutagenesis), oligonucleotide-mediated (or site-directed) mutagenesis, PCR mutagenesis and cassette mutagenesis of an earlier prepared variant or non-variant version of an isolated natural promoter according to the invention.

Oligonucleotide-mediated mutagenesis is a preferred method for preparing nucleotide substitution variants of a polynucleotide of the invention. This technique is well known in the art as, for example, described by Adelman et al. (1983, DNA 2:183). Briefly, a polynucleotide according to any one of SEQ ID NO: 1, 3, 5 or 7 is altered by hybridising an oligonucleotide encoding the desired mutation to a template DNA, wherein the template is the single-stranded form of a plasmid or bacteriophage containing the unaltered or parent DNA sequence. After hybridisation, a DNA polymerase is used to synthesise an entire second complementary strand of the template that will thus incorporate the oligonucleotide primer, and will code for the selected alteration in said parent DNA sequence.

Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridise properly to the single-stranded DNA template molecule.

The DNA template can be generated by those vectors that are either derived from bacteriophage M13 vectors, or those vectors that contain a single-stranded phage origin of replication as described by Viera et al. (1987, Methods Enzymol. 153:3). Thus, the DNA that is to be mutated may be inserted into one of the vectors to generate single-stranded template. Production of single-stranded template is described, for example, in Sections 4.21-4.41 of Sambrook et al. (1989, supra).

Alternatively, the single-stranded template may be generated by denaturing double-stranded plasmid (or other DNA) using standard techniques.

For alteration of the native DNA sequence, the oligonucleotide is hybridised to the single-stranded template under suitable hybridisation conditions. A DNA polymerising enzyme, usually the Klenow fragment of DNA polymerase 1, is then added to synthesise the complementary strand of the template using the oligonucleotide as a primer for synthesis. A heteroduplex molecule is thus formed such that one strand of DNA encodes the mutated form of the polypeptide or fragment under test, and the other strand (the original template) encodes the native unaltered sequence of the polypeptide or fragment under test. This heteroduplex molecule is then transformed into a suitable host cell, usually a prokaryote such as E. coli. After the cells are grown, they are plated onto agarose plates and screened using the oligonucleotide primer having a detectable label to identify the bacterial colonies having the mutated DNA. The resultant mutated DNA fragments are then cloned into suitable expression hosts such as E. coli using conventional technology and clones that retain the desired antigenic activity are detected. Where the clones have been derived using random mutagenesis techniques, positive clones would have to be sequenced in order to detect the mutation.

Alternatively, linker-scanning mutagenesis of DNA may be used to introduce clusters of point mutations throughout a sequence of interest that has been cloned into a plasmid vector. For example, reference may be made to Ausubel et al., supra, (in particular, Chapter 8.4) which describes a first protocol that uses complementary oligonucleotides and requires a unique restriction site adjacent to the region that is to be mutagenised. A nested series of deletion mutations is first generated in the region. A pair of complementary oligonucleotides is synthesised to fill in the gap in the sequence of interest between the linker at the deletion endpoint and the nearby restriction site. The linker sequence actually provides the desired clusters of point mutations as it is moved or “scanned” across the region by its position at the varied endpoints of the deletion mutation series. An alternate protocol is also described by Ausubel et al., supra, which makes use of site directed mutagenesis procedures to introduce small clusters of point mutations throughout the target region. Briefly, mutations are introduced into a sequence by annealing a synthetic oligonucleotide containing one or more mismatches to the sequence of interest cloned into a single-stranded M13 vector. This template is grown in an E. coli dut⁻ ung⁻ strain, which allows the incorporation of uracil into the template strand. The oligonucleotide is annealed to the template and extended with T4 DNA polymerase to create a double-stranded heteroduplex. Finally, the heteroduplex is introduced into a wild-type E. coli strain, which will prevent replication of the template strand due to the presence of apurinic sites (generated where uracil is incorporated), thereby resulting in plaques containing only mutated DNA.

Region-specific mutagenesis and directed mutagenesis using PCR may also be employed to construct polynucleotide variants according to the invention. In this regard, reference may be made, for example, to Ausubel et al., supra, in particular Chapters 8.2A and 8.5.

Alternatively, suitable polynucleotide sequence variants of the invention may be prepared according to the following procedure:

(a) creating primers which are optionally degenerate wherein each comprises a portion of a reference polynucleotide encoding a reference polypeptide or fragment of the invention, preferably encoding the sequence set forth in any one of SEQ ID NO: 2, 4 or 8;

(b) obtaining a nucleic acid extract from a bacterial species, which is preferably of a genus selected from the group consisting of Aeromonas, Vibrio and Edwardsiella; and

(c) using said primers to amplify, via nucleic acid amplification techniques, at least one amplification product from said nucleic acid extract, wherein said amplification product corresponds to a polynucleotide variant.

Suitable nucleic acid amplification techniques are well known to the skilled addressee, and include polymerase chain reaction (PCR) as for example described in Ausubel et al. (supra); strand displacement amplification (SDA) as for example described in U.S. Pat. No. 5,422,252; rolling circle replication (RCR) as for example described in Liu et al., (1996, J. Am. Chem. Soc. 118:1587-1594 and International application WO 92/01813) and Lizardi et al., (International Application WO 97/19193); nucleic acid sequence-based amplification (NASBA) as for example described by Sooknanan et al., (1994, Biotechniques 17:1077-1080); and Q-replicase amplification as for example described by Tyagi et al., (1996, Proc. Natl. Acad. Sci. USA 93: 5395-5400).

Typically, polynucleotide variants that are substantially complementary to a reference polynucleotide are identified by blotting techniques that include a step whereby nucleic acids are immobilised on a matrix (preferably a synthetic membrane such as nitrocellulose), followed by a hybridisation step, and a detection step. Southern blotting is used to identify a complementary DNA sequence; northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al. (1994-1998, supra) at pages 2.9.1 through 2.9.20.

According to such methods, Southern blotting involves separating DNA molecules according to size by gel electrophoresis, transferring the size-separated DNA to a synthetic membrane, and hybridising the membrane-bound DNA to a complementary nucleotide sequence labelled radioactively, enzymatically or fluorochromatically. In dot blotting and slot blotting, DNA samples are directly applied to a synthetic membrane prior to hybridisation as above.

An alternative blotting step is used when identifying complementary polynucleotides in a cDNA or genomic DNA library, such as through the process of plaque or colony hybridisation. A typical example of this procedure is described in Sambrook et al. (“Molecular Cloning. A Laboratory Manual”, Cold Spring Harbour Press, 1989) Chapters 8-12.

Typically, the following general procedure can be used to determine hybridisation conditions. Polynucleotides are blotted/transferred to a synthetic membrane, as described above. A reference polynucleotide such as a polynucleotide of the invention is labelled as described above, and the ability of this labelled polynucleotide to hybridise with an immobilised polynucleotide is analysed.

A skilled addressee will recognise that a number of factors influence hybridisation. The specific activity of radioactively labelled polynucleotide sequence should typically be greater than or equal to about 108 dpm/mg to provide a detectable signal. A radiolabelled nucleotide sequence of specific activity 108 to 109 dpm/mg can detect approximately 0.5 pg of DNA. It is well known in the art that sufficient DNA must be immobilised on the membrane to permit detection. It is desirable to have excess immobilised DNA, usually 10 g. Adding an inert polymer such as 10% (w/v) dextran sulfate (MW 500,000) or polyethylene glycol 6000 during hybridisation can also increase the sensitivity of hybridisation (see Ausubel supra at 2.10.10).

To achieve meaningful results from hybridisation between a polynucleotide immobilised on a membrane and a labelled polynucleotide, a sufficient amount of the labelled polynucleotide must be hybridised to the immobilised polynucleotide following washing. Washing ensures that the labelled polynucleotide is hybridised only to the immobilised polynucleotide with a desired degree of complementarity to the labelled polynucleotide.

It will be understood that polynucleotide variants according to the invention will hybridise to a reference polynucleotide under at least low stringency conditions. Reference herein to low stringency conditions include and encompass from at least about 1% v/v to at least about 15% v/v formamide and from at least about 1 M to at least about 2 M salt for hybridisation at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridisation at 65° C., and (i) 2×SSC, 0.1% SDS; or (ii) 0.5% BSA, 1 mM EDTA, 40 mM NaHPO₄ (pH 7.2), 5% SDS for washing at room temperature.

Suitably, the polynucleotide variants hybridise to a reference polynucleotide under at least medium stringency conditions. Medium stringency conditions include and encompass from at least about 16% v/v to at least about 30% v/v formamide and from at least about 0.5 M to at least about 0.9 M salt for hybridisation at 42° C., and at least about 0.5 M to at least about 0.9 M salt for washing at 42° C. Medium stringency conditions also may include 1% Bovine Serum Albumin (BSA), 1 mM EDTA, 0.5 M NaHPO₄ (pH 7.2), 7% SDS for hybridisation at 65° C., and (i) 2×SSC, 0.1% SDS; or (ii) 0.5% BSA, 1 mM EDTA, 40 mM NaHPO₄ (pH 7.2), 5% SDS for washing at 42° C.

Preferably, the polynucleotide variants hybridise to a reference polynucleotide under high stringency conditions. High stringency conditions include and encompass from at least about 31% v/v to at least about 50% v/v formamide and from at least about 0.01 M to at least about 0.15 M salt for hybridisation at 42° C., and at least about 0.01 M to at least about 0.15 M salt for washing at 42° C. High stringency conditions also may include 1% BSA, 1 mM EDTA, 0.5 M NaHPO₄ (pH 7.2), 7% SDS for hybridisation at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C.

Other stringent conditions are well known in the art. A skilled addressee will recognise that various factors can be manipulated to optimise the specificity of the hybridisation. Optimisation of the stringency of the final washes can serve to ensure a high degree of hybridisation. For detailed examples, see Ausubel et al., supra at pages 2.10.1 to 2.10.16 and Sambrook et al. (1989, supra) at sections 1.101 to 1.104.

While stringent washes are typically carried out at temperatures from about 42° C. to 68° C., one skilled in the art will appreciate that other temperatures may be suitable for stringent conditions. Maximum hybridisation typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel et al., supra at page 2.10.8).

In general, washing is carried out at T=69.3+0.41 (G+C) %-12° C. However, the Tₘ of a duplex DNA decreases by 1° C. with every increase of 1% in the number of mismatched base pairs.

In a preferred hybridisation procedure, a membrane (e.g., a nitrocellulose membrane or a nylon membrane) containing immobilised DNA is hybridised overnight at 42° C. in a hybridisation buffer (50% deionised formamide, 5×SSC, 5× Denhardt's solution (0.1% ficoll, 0.1% polyvinylpyrollidone and 0.1% bovine serum albumin), 0.1% SDS and 200 mg/mL denatured salmon sperm DNA) containing labelled probe. The membrane is then subjected to two sequential medium stringency washes (i.e., 2×SSC/0.1% SDS for 15 min at 45° C., followed by 2×SSC/0.1% SDS for 15 min at 50° C.), followed by two sequential high stringency washes (i.e., 0.2×SSC/0.1% SDS for 12 min at 55° C. followed by 0.2×SSC and 0.1% SDS solution for 12 min).

Methods for detecting a labelled polynucleotide hybridised to an immobilised polynucleotide are well known to practitioners in the art. Such methods include autoradiography, phosphorimaging, and chemiluminescent, fluorescent and calorimetric detection.

3.3 Oligonucleotides

The invention also relates to oligonucleotides which specifically hybridize to a polynucleotide of the invention or its complement. The term “specifically hybridize” means that such an oligonucleotide hybridizes under stringent conditions specifically to a polynucleotide of the invention or its complement. The oligonucleotides may be used as probes or as primers in nucleic acid based detection as described, infra. The length of the oligonucleotides will vary depending on its use, for example, the length will be typically 15 to 35 nucleotides if the oligonucleotides are used as primers. Such oligonucleotides can be synthesized using conventional synthesis techniques.

4. Antigen-Binding Molecules

The invention also contemplates antigen-binding molecules against the aforementioned polypeptides, fragments, variants and derivatives. For example, the antigen-binding molecules may comprise whole polyclonal antibodies. Such antibodies may be prepared, for example, by injecting a polypeptide, fragment, variant or derivative of the invention into a production species, which may include mice or rabbits, to obtain polyclonal antisera. Methods of producing polyclonal antibodies are well known to those skilled in the art. Exemplary protocols which may be used are described for example in Coligan et al., CURRENT PROTOCOLS IN IMMUNOLOGY, (John Wiley & Sons, Inc, 1991), and Ausubel et al., (1994-1998, supra), in particular Section III of Chapter 1.

In lieu of the polyclonal antisera obtained in the production species, monoclonal antibodies may be produced using the standard method as described, for example, by Köhler and Milstein (1975, Nature 256, 495-497), or by more recent modifications thereof as described, for example, in Coligan et al., (1991, supra) by immortalising spleen or other antibody producing cells derived from a production species which has been inoculated with one or more of the polypeptides, fragments, variants or derivatives of the invention.

The invention also contemplates as antigen-binding molecules Fv, Fab, Fab′ and F(ab′)₂ immunoglobulin fragments.

Alternatively, the antigen-binding molecule may comprise a synthetic stabilised Fv fragment. Exemplary fragments of this type include single chain Fv fragments (sFv, frequently termed scFv) in which a peptide linker is used to bridge the N terminus or C terminus of a V_{H} domain with the C terminus or N-terminus, respectively, of a V_{L} domain. ScFv lack all constant parts of whole antibodies and are not able to activate complement. Suitable peptide linkers for joining the V_{H} and V_{L} domains are those which allow the V_{H} and V_{L} domains to fold into a single polypeptide chain having an antigen binding site with a three dimensional structure similar to that of the antigen binding site of a whole antibody from which the Fv fragment is derived. Linkers having the desired properties may be obtained by the method disclosed in U.S. Pat. No. 4,946,778. However, in some cases a linker is absent. ScFvs may be prepared, for example, in accordance with methods outlined in Kreber et al (Krebber et al. 1997, J Immunol. Methods; 201(1): 35-55). Alternatively, they may be prepared by methods described in U.S. Pat. No. 5,091,513, European Patent No 239,400 or the articles by Winter and Milstein (1991, Nature 349:293) and Plückthun et al (1996, In Antibody engineering: A practical approach. 203-252).

Alternatively, the synthetic stabilised Fv fragment comprises a disulphide stabilised Fv (dsFv) in which cysteine residues are introduced into the V_{H} and V_{L} domains such that in the fully folded Fv molecule the two residues will form a disulphide bond therebetween. Suitable methods of producing dsFv are described for example in Glockscuther et al. Biochem. 29: 1363-1367; Reiter et al. 1994, J. Biol. Chem. 269: 18327-18331; Reiter et al. 1994, Biochem. 33: 5451-5459; Reiter et al. 1994. Cancer Res. 54: 2714-2718; Webber et al. 1995, Mol. Immunol. 32: 249-258.

Also contemplated as antigen-binding molecules are single variable region domains (termed dAbs) as for example disclosed in (Ward et al. 1989, Nature 341: 544-546; Hamers-Casterman et al. 1993, Nature. 363: 446-448; Davies & Riechmann, 1994, FEBS Lett. 339: 285-290).

Alternatively, the antigen-binding molecule may comprise a “minibody”. In this regard, minibodies are small versions of whole antibodies, which encode in a single chain the essential elements of a whole antibody. Suitably, the minibody is comprised of the V_{H} and V_{L} domains of a native antibody fused to the hinge region and CH3 domain of the immunoglobulin molecule as, for example, disclosed in U.S. Pat. No. 5,837,821.

In an alternate embodiment, the antigen binding molecule may comprise non-immunoglobulin derived, protein frameworks. For example, reference may be made to (Ku & Schultz, 1995, Proc. Natl. Acad. Sci. USA, 92: 652-6556) which discloses a four-helix bundle protein cytochrome b562 having two loops randomised to create complementarity determining regions (CDRs), which have been selected for antigen binding.

The antigen-binding molecule may be multivalent (i.e., having more than one antigen binding site). Such multivalent molecules may be specific for one or more antigens. Multivalent molecules of this type may be prepared by dimerisation of two antibody fragments through a cysteinyl-containing peptide as, for example disclosed by (Adams et al., 1993, Cancer Res. 53: 4026-4034; Cumber et al., 1992, J. Immunol. 149: 120-126). Alternatively, dimerisation may be facilitated by fusion of the antibody fragments to amphiphilic helices that naturally dimerise (Pack P. Plηnckthun, 1992, Biochem. 31: 1579-1584), or by use of domains (such as the leucine zippers jun and fos) that preferentially heterodimerise (Kostelny et al., 1992, J. Immunol. 148: 1547-1553). In an alternate embodiment, the multivalent molecule may comprise a multivalent single chain antibody (multi-scFv) comprising at least two scFvs linked together by a peptide linker. In this regard, non-covalently or covalently linked scFv dimers termed “diabodies” may be used. Multi-scFvs may be bispecific or greater depending on the number of scFvs employed having different antigen binding specificities. Multi-scFvs may be prepared for example by methods disclosed in U.S. Pat. No. 5,892,020.

The antigen-binding molecules of the invention may be used for affinity chromatography in isolating a natural or recombinant polypeptide or immuno-interactive fragment of the invention. For example reference may be made to immunoaffinity chromatographic procedures described in Chapter 9.5 of Coligan et al., (1995-1997, supra).

The antigen-binding molecules can be used to screen expression libraries for variant polypeptides of the invention as described herein. They can also be used to detect polypeptides, fragments, variants and derivatives of the invention as described hereinafter.

5. Methods of Detection

5.1. Protein-Based Detection

The invention also extends to a method of detecting in a sample a polypeptide, fragment, variant or derivative as broadly described above, comprising contacting the sample with an antigen-binding molecule as described in Section 4 and detecting the presence of a complex comprising the antigen-binding molecule and the polypeptide, fragment, variant or derivative in said contacted sample. The presence of the complex in the sample is indicative of a bacterial infection and the antigen-binding molecule of the present invention, including antibodies thus may be used to detect or diagnose bacterial infection. The invention also provides a method of detecting or diagnosing a bacterial infection of an animal comprising contacting a sample from an animal with a polypeptide according to the invention and determining the presence or absence of a complex between the polypeptide and antibodies specific to the polypeptide. The presence of the complex is indicative of an infection. In one embodiment, the bacterial infection detected by these methods is of the genus Aeromonas, Vibrio or Edwardsiella.

Any suitable technique for determining formation of the complex may be used. For example, an antigen-binding molecule according to the invention, having a reporter molecule associated therewith may be utilised in immunoassays. Such immunoassays include, but are not limited to, radioimmunoassays (RIAs), enzyme-linked immunosorbent assays (ELISAs) and immunochromatographic techniques (ICTs), Western blotting which are well known those of skill in the art. For example, reference may be made to “CURRENT PROTOCOLS IN IMMUNOLOGY” (1994, supra) which discloses a variety of immunoassays that may be used in accordance with the present invention. Immunoassays may include competitive assays as understood in the art or as for example described infra. It will be understood that the present invention encompasses qualitative and quantitative immunoassays.

Suitable immunoassay techniques are described for example in U.S. Pat. Nos. 4,016,043, 4, 424,279 and 4,018,653. These include both single-site and two-site assays of the non-competitive types, as well as the traditional competitive binding assays. These assays also include direct binding of a labelled antigen-binding molecule to a target antigen.

Two site assays are particularly favoured for use in the present invention. A number of variations of these assays exist, all of which are intended to be encompassed by the present invention. Briefly, in a typical forward assay, an unlabelled antigen-binding molecule such as an unlabelled antibody is immobilised on a solid substrate and the sample to be tested brought into contact with the bound molecule. After a suitable period of incubation, for a period of time sufficient to allow formation of an antibody-antigen complex, another antigen-binding molecule, suitably a second antibody specific to the antigen, labelled with a reporter molecule capable of producing a detectable signal is then added and incubated, allowing time sufficient for the formation of another complex of antibody-antigen-labelled antibody. Any unreacted material is washed away and the presence of the antigen is determined by observation of a signal produced by the reporter molecule. The results may be either qualitative, by simple observation of the visible signal, or may be quantitated by comparing with a control sample containing known amounts of antigen. Variations on the forward assay include a simultaneous assay, in which both sample and labelled antibody are added simultaneously to the bound antibody. These techniques are well known to those skilled in the art, including minor variations as will be readily apparent. In accordance with the present invention, the sample is one that might contain an antigen including serum, whole blood, and plasma or lymph fluid. The sample is, therefore, generally a circulatory sample comprising circulatory fluid.

In the typical forward assay, a first antibody having specificity for the antigen or antigenic parts thereof is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well known in the art and generally consist of cross-linking covalently binding or physically adsorbing, the polymer-antibody complex is washed in preparation for the test sample. An aliquot of the sample to be tested is then added to the solid phase complex and incubated for a period of time sufficient and under suitable conditions to allow binding of any antigen present to the antibody. Following the incubation period, the antigen-antibody complex is washed and dried and incubated with a second antibody specific for a portion of the antigen. The second antibody has generally a reporter molecule associated therewith that is used to indicate the binding of the second antibody to the antigen. The amount of labelled antibody that binds, as determined by the associated reporter molecule, is proportional to the amount of antigen bound to the immobilised first antibody.

An alternative method involves immobilising the antigen in the biological sample and then exposing the immobilised antigen to specific antibody that may or may not be labelled with a reporter molecule. Depending on the amount of target and the strength of the reporter molecule signal, a bound antigen may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

From the foregoing, it will be appreciated that the reporter molecule associated with the antigen-binding molecule may include the following:

(a) direct attachment of the reporter molecule to the antigen-binding molecule;

(b) indirect attachment of the reporter molecule to the antigen-binding molecule; i.e., attachment of the reporter molecule to another assay reagent which subsequently binds to the antigen-binding molecule; and

(c) attachment to a subsequent reaction product of the antigen-binding molecule.

The reporter molecule may be selected from a group including a chromogen, a catalyst, an enzyme, a fluorochrome, a chemiluminescent molecule, a lanthamide ion such as Europium (Eu³⁴), a radioisotope and a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes suitable for use as reporter molecules is disclosed in United States Patent Specifications U.S. Pat. No. 4,366,241, U.S. Pat. No. 4,843,000, and U.S. Pat. No. 4,849,338. Suitable enzymes useful in the present invention include alkaline phosphatase, horseradish peroxidase, luciferase, -galactosidase, glucose oxidase, lysozyme, malate dehydrogenase and the like. The enzymes may be used alone or in combination with a second enzyme that is in solution.

Suitable fluorochromes include, but are not limited to, fluorescein isothiocyanate (FITC), tetramethylrhodamine isothiocyanate (TRITC), R-Phycoerythrin (RPE), and Texas Red. Other exemplary fluorochromes include those discussed by Dower et al. (International Publication WO 93/06121). Reference also may be made to the fluorochromes described in U.S. Pat. No. 5,573,909 (Singer et al), U.S. Pat. No. 5,326,692 (Brinkley et al). Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218.

In the case of an enzyme immunoassay, an enzyme is conjugated to the second antibody, generally by means of glutaraldehyde or periodate. As will be readily recognised, however, a wide variety of different conjugation techniques exist which are readily available to the skilled artisan. The substrates to be used with the specific enzymes are generally chosen for the production of, upon hydrolysis by the corresponding enzyme, a detectable colour change. Examples of suitable enzymes include those described supra. It is also possible to employ fluorogenic substrates, which yield a fluorescent product rather than the chromogenic substrates noted above. In all cases, the enzyme-labelled antibody is added to the first antibody-antigen complex. It is then allowed to bind, and excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample.

Alternately, fluorescent compounds, such as fluorescein, rhodamine and the lanthamide, europium (EU), may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. The fluorescent-labelled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (IFMA) are well established in the art. However, other reporter molecules, such as radioisotope, chemiluminescent or bioluminescent molecules may also be employed.

5.2. Nucleic acid-based detection

In another aspect, the invention provides a method of detecting a bacterial species, which is preferably of a genus selected from the group consisting of Aeromonas Vibrio, or Edwardsiella, in a biological sample suspected of containing said bacteria. The method comprises isolating the biological sample from an animal, detecting a nucleic acid sequence according to the invention in said sample which indicates the presence of said bacteria.

Detection of the said nucleic acid sequence may be determined using any suitable technique. For example, a labelled nucleic acid sequence according to the invention may be used as a probe in a Southern blot of a nucleic acid extract obtained from an animal as is well known in the art. Alternatively, a labelled nucleic acid sequence according to the invention may be utilised as a probe in a Northern blot of a RNA extract from the patient. Preferably, a nucleic acid extract from the animal is utilised in concert with oligonucleotide primers corresponding to sense and antisense sequences of a nucleic acid sequence according to the invention, or flanking sequences thereof, in a nucleic acid amplification reaction such as PCR, or the ligase chain reaction (LCR). A variety of automated solid-phase detection techniques are also appropriate. For example, very large scale immobilised primer arrays (VLSIPS™) are used for the detection of nucleic acids as for example described by Fodor et al., (1991, Science 251:767-777) and Kazal et al., (1996, Nature Medicine 2:753-759). The above generic techniques are well known to persons skilled in the art.

6. Compositions

The invention also encompasses a composition comprising a polypeptide, variant or derivative as broadly described above (“immunogenic agents”), together with a pharmaceutically acceptable carrier which may be administered to elicit an immune response in an animal, preferably a freshwater and/or marine animal, more preferably a fish, which response includes production of elements that protect said animal against infection by a bacterial species which is preferably of a genus selected from the group consisting of Aeromonas, Vibrio and Edwardsiella or that specifically bind to said agents or to said bacterial species. Optionally, said composition further comprises an adjuvant.

In one embodiment, the composition is adminstered to an animal intraperitoneally, or by spraying the animal with the composition or by immersion of the animal in said composition.

A further feature of the invention is the use of the antigen-binding molecules of the invention (“therapeutic agents”) as actives, together with a pharmaceutically acceptable carrier, in a composition for protecting or treating an animal against a condition associated with a bacterial species expressing a polypeptide or polypeptide variant according to the invention. The species is preferably of a genus selected from the group consisting of Aeromonas, Vibrio and Edwardsiella. Preferably, the condition is selected from the group consisting of fish motile aeromonad septicemia, Vibriosis and Edwardsiellosis.

Depending upon the particular route of administration, a variety of pharmaceutically acceptable carriers, well known in the art may be used. These carriers may be selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Any suitable route of administration may be employed for providing a mammal or a patient with a composition of the invention. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed. Intra-muscular and subcutaneous injection is appropriate, for example, for administration of immunogenic compositions, vaccines and DNA vaccines.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of an immunogenic or a therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be effected by using other polymer matrices, liposomes and/or microspheres.

Compositions suitable for oral or parenteral administration may be presented as discrete units such as capsules, sachets or tablets each containing a pre-determined amount of one or more immunogenic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more immunogenic agents as described above with the carrier which constitutes one or more necessary ingredients. In general, the compositions are prepared by uniformly and intimately admixing the immunogenic agents of the invention with liquid carriers or finely divided solid carriers or both, and then, if necessary, shaping the product into the desired presentation.

The above compositions may be administered in a manner compatible with the dosage formulation, and in such amount as is therapeutically effective or immunogenically effective as the case may be. In this regard, the dose of immunogenic agent administered to an animal should be sufficient to elicit an immune response that includes the production of elements that protect said animal against infection by a said bacterial species. For fish, about 1.5 to about 3.0 ug of polypeptide of the invention per gram of fish body weight may be administered. In one embodiment, about 1.5 ug per gram of fish body weight is administered.

Alternatively, the dose of therapeutic agent administered to a patient should be sufficient to effect a beneficial response in the patient over time such as a reduction in the level of a said bacterial species or to ameliorate the condition to be treated. In general, the quantity of the therapeutic agent(s) to be administered may depend on the subject to be treated inclusive of the age, sex, weight and general health condition thereof. In this regard, precise amounts of the therapeutic agent(s) for administration will depend on the judgement of the practitioner. In determining the effective amount of the therapeutic agent to be administered in the treatment or prophylaxis of a condition associated with infection by a said bacterial species, the physician may evaluate circulating plasma levels, progression of the condition, and the production of antibodies against a polypeptide, fragment, variant or derivative of the invention.

In any event, those of skill in the art may readily determine suitable dosages of the immunogenic and therapeutic agents of the invention. Such dosages may be in the order of nanograms to milligrams of the immunogenic agents of the invention.

An immunogenic agent according to the invention can be mixed, conjugated or fused with other antigens, including B or T cell epitopes of other antigens. In addition, it can be conjugated to a carrier as described below.

When an haptenic peptide is used (i.e., a peptide which reacts with cognate antibodies, but cannot itself elicit an immune response), it can be conjugated with an immunogenic carrier. Useful carriers are well known in the art and include for example: thyroglobulin; albumins such as human serum albumin; toxins, toxoids or any mutant crossreactive material (CRM) of the toxin from tetanus, diphtheria, pertussis, Pseudomonas, E. coli, Staphylococcus, and Streptococcus; polyamino acids such as poly(lysine:glutamic acid); influenza; Rotavirus VP6, Parvovirus VP1 and VP2; hepatitis B virus core protein; hepatitis B virus recombinant vaccine and the like. Alternatively, a fragment or epitope of a carrier protein or other immunogenic protein may be used. For example, an haptenic peptide can be coupled to a T cell epitope of a bacterial toxin, toxoid or CRM. In this regard, reference may be made to U.S. Pat. No. 5,785,973.

The immunogenic compositions may include an adjuvant as is well known in the art. Suitable adjuvants include, but are not limited to: surface active substances such as hexadecylamine, octadecylamine, octadecyl amino acid esters, lysolecithin, dimethyldioctadecylammonium bromide, N,N-dicoctadecyl-N′,N′bis(2-hydroxyethyl-propanediamine), methoxyhexadecylglycerol, and pluronic polyols; polyamines such as pyran, dextransulfate, poly IC carbopol; peptides such as muramyl dipeptide and derivatives, dimethylglycine, tuftsin; oil emulsions; and mineral gels such as aluminum phosphate, aluminum hydroxide or alum; lymphokines, and QuilA.

In a further embodiment, a polynucleotide of the invention may be used as an immunogenic composition in the form of a “naked DNA” composition as is known in the art. For example, an expression vector of the invention may be introduced into an animal, where it causes production of a polypeptide, fragment, variant or derivative according to the invention in vivo, against which the host mounts an immune response as for example described in Barry, M. et al., (1995, Nature, 377:632-635).

7., Detection Kits

The present invention also provides kits for the detection in a biological sample of a bacterial species which is preferably of a genus selected from the group consisting of Aeromonas, Vibrio or Edwardsiella. These will contain one or more particular agents described above depending upon the nature of the test method employed. In this regard, the kits may include one or more of a polypeptide, fragment, variant, derivative, antigen-binding molecule or nucleic acid according to the invention. The kits may also optionally include appropriate reagents for detection of labels, positive and negative controls, washing solutions, dilution buffers and the like. For example, a nucleic acid-based detection kit may include (i) a nucleic acid according to the invention (which may be used as a positive control), (ii) an oligonucleotide primer according to the invention, and optionally a DNA polymerase, DNA ligase etc depending on the nucleic acid amplification technique employed.

The present invention is further described with reference to the following non-limiting examples.

### EXAMPLE 1

Isolation and Characterisation of a 43-kDa Major Adhesin from A. hydrophila

Materials and Methods

Bacterial Strains Phase, Plasmids and Media

A. hydrophila PPD 134/91 was obtained from the Primary Production department, Singapore. It was routinely cultured in tryptic soy broth (TSB, Difco, USA) or on tryptic soy agar (TSA) at 25° C. Escherichia coli JM 109 (Promega, USA) was used as the host for recombinant plasmids during cloning, sub-cloning and sequencing studies. E. coli XL1-Blue MRF′ (Stratagene, La Jolla, Calif., USA) was used as the host for XZAP Express phage. E. coli M 15 (QIAGEN, Valencia, Calif., USA) was used as the host for expression of cloned gene. Plasmid pGEM-T (Promega, USA) was used as the PCR cloning vector and for sequencing. Phage ZAP Express and XZAP II (Stratagene) was used to construct genomic DNA libraries. Plasmid pQE-30 (QIAGEN, USA) was used as the expression vector in E. coli. E. coli bacteria were grown in Luria-Bertani (LB) broth (DIFCO, USA) or on LB agar at 37° C. When required, the medium was supplemented with 100 μg mL⁻¹ ampicillin and/or kanamycin (15 μg mL⁻¹).

DNA Preparation, Manipulation and Analysis

Small-scale plasmid preparations were performed using the Wizard Mini Preps DNA purification kit (Promega, USA). A. hydrophila PPD 134/91 chromosomal DNA was extracted by the QIAGEN Genomic-tip 500/G column (QIAGEN, USA). Other DNA routine manipulations were performed as described by Sambrook et al. MOLECULAR CLONING, A LABORATORY MANUAL. 2^{nd} edition, Cold Spring Harbor laboratory Press, New York, (1989).

Construction of A. hydrophila Genomic Library

Two genomic libraries were constructed. For Library 1: the chromosomal DNA from A. hydrophila PPD 134/91 was partially digested using the restriction endonuclease Sau3AI. Digestion condition was first optimised on a small scale to get an extent of digestion ranging from 1 kb to 10 kb. Digested DNA was purified by phenol-chloroform extraction. 125 ng DNA was ligated to the BamHI-predigested and CLAP-treated ZAP Express phage vector (Stratagene). Then, 50 ng of ligated DNA was packaged into lambda phage heads using the Gigapack™ III Gold kit as described by the manufacturer (Stratagene). The resulting phage was transduced into E. coli XL-1-Blue MRF′ and plated selectively on LB agar containing tetracycline and X-gal. Inserts were present in >90% of the transductants. The library was later amplified for once. For Library 2: the chromosomal DNA was completely digested using EcoRI at 37° C. for 3 hr. Digested DNA fragments were separated on a 0.5% agarose gel, and those DNA bands ranging from 5-10 Kb were excised from the gel and purified using the Pre-A-Gene™ DNA Purification Kit (Bio-Rad, USA). 100 ng purified DNA was ligated to the EcoRI pre-digested and CIAP-treated % ZAP II vector (Stratagene). Then, 50 ng of ligated DNA was packaged and amplified as Library 1.

Cloning of the Gene Encoding the 43-kDa Protein

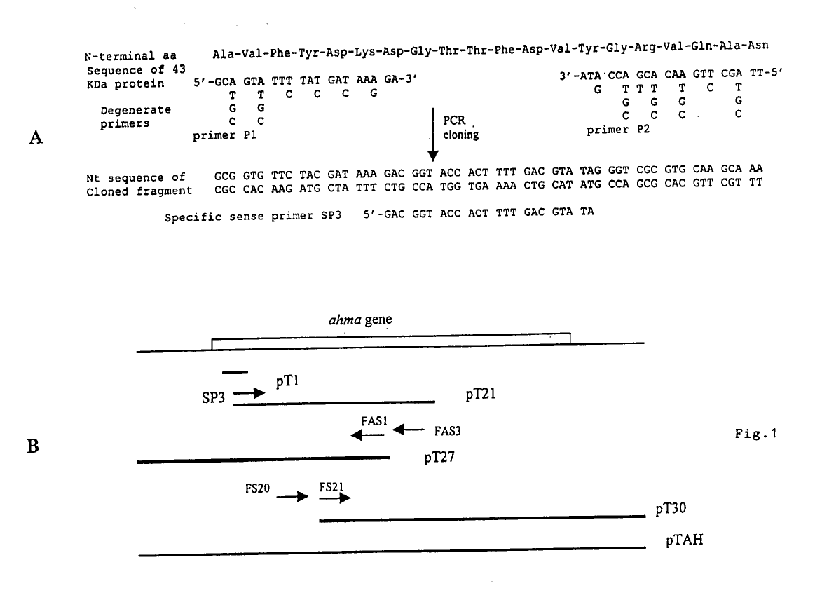

Degenerate primers P1 and P2 (shown in FIG. 1, panel A) were designed on the basis of the N-terminal 20-amino acid sequence of the 43-kDa protein and were used for PCR amplification in a reaction containing 5 units Taq DNA polymerase, 1.25 mM of each dNTP, 1 pmol of each primer, and 100 ng template DNA, in a total volume of 50 μL. A. hydrophila PPD 134/91 chromosomal DNA was used as a template DNA after partially digestion by Sau3AI as described above. PCR products were electrophoresed on a 3% agarose gel and a 59 bp DNA band was purified. Purified DNA was ligated to the plasmid pGEM-T™ vector and then introduced into competent E. coli JM 109 cells according to the manufacturer's instruction (Promega, USA). The resultant plasmid (pTI) was sequenced and the nucleotide sequence information was used to design a specific sense primer SP3 as shown in FIG. 1 in a 3′ nested RACE PCR using genomic library 1 as template (1 μL of the amplified phage). A 650 bp DNA fragment was cloned (pT21) and its nucleotide sequence determined. This analysis revealed a gene encoding the N-terminal amino acid sequence. Then, specific sense and antisense primers were constructed from this 650 bp DNA fragment and used for 3′ RACE and 5′ RACE nested PCR using either library 1 or library 2 as template. The full length of the major adhesin gene (designated as ahma) was then cloned by nested specific PCR.

DNA Sequencing

DNA sequencing was performed using Big Dye Terminator Cycle Sequencing Ready Reaction kit and protocol developed by Applied Biosystems (Perkin-Elmer Corp., Foster City. Calif., USA). The labelled extension products were analysed on a model 373A DNA sequencer (Applied Biosystems). The nucleotide (nt) and deduced amino acid (aa) sequences were analysed using DNASIS programs.

Results

The N-terminal amino acid sequence disclosed in Lee, et al. (1997, supra) was used to design two degenerate primers P1 and P2 for PCR reaction (FIG. 1, panel A). After PCR using these degenerate primers, one 59 bp dominant DNA fragments was sub-cloned (plasmid pTI) and sequenced. The aa sequence deduced from this 59 bp fragment corresponded to the N-terminal sequence of the known 43 kDa protein. A specific sense primer SP3 was constructed according to the central region of this 59 bp fragment and used for 3′ nested RACE PCR using genomic library 1 as template. Agarose gel electrophoresis of the PCR products revealed several bands. After Southern hybridisation with an oligonucleotide probe (as same as primer SP3), a 650 bp fragment was selected, sub-cloned (plasmid pT21) and sequenced. This fragment was confirmed as part of the 43-kDa protein gene by comparing a part of 21 nt sequence corresponding to the 7 C-terminal amino acids of the known N-terminal aa sequence.

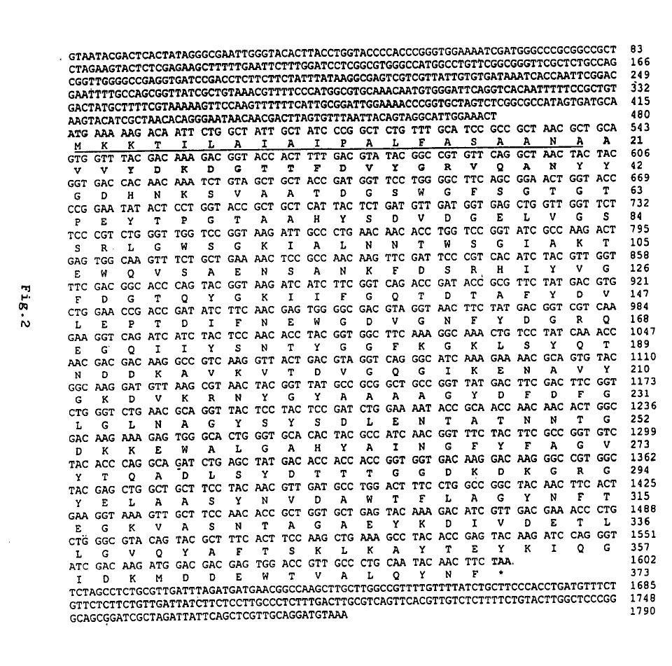

Specific primers generated from the 650 bp partial adhesin gene clone were used in 5′RACE and 3′RACE nested PCR reactions to isolate the full-length major adhesin gene from the genomic libraries. The anti-sense primers are: FASI: 5′-GGT CGG TTC CAG CAC GTC AT-3′ and FAS3: 5′-ACC TAC GTC GCC CCA CTC GTT GAA G-3′ for 5′RACE; and sense primer primers: FS20: 5′-AGC TGC TAC CGA TGG TTC CTG GGG-3′ and FS21: 5′-AAA CTC CGC CAA CAA GTT CG-3′ for 3′RACE. A 1.1 kb 3′RACE PCR product was confirmed to encode the 3′ end of the ahma gene (pT30). A 900 bp 5′RACE PCR product was also confirmed to encode the 5′ end of the ahma gene (pT27, FIG. 1, panel B). Together, the two overlapping partial clones comprised a 1790 bp DNA which contains the full-length A. hydrophila major adhesin coding region of 1119 bp (FIG. 2) encoding a putative precursor protein of 373 amino acids containing a putative 20 amino acid signal peptide and a mature major adhesin protein, which is predicted to comprise 353 amino acids (Mr 38.7 kDa). The predicted 20 contiguous N-terminal amino acids are identical to those obtained by Edman degradation except that the third amino acid is valine instead of phenylalanine. The inventors suspect that this discrepancy is a result of misreading the Edman degradation results. Using two specific primers, the 1810 bp DNA fragment was cloned by PCR from the chromosomal DNA of A. hydrophila PPD 134/91 and designated as pTAH (FIG. 1, panel B).

The homology between the deduced polypeptide sequence and that of other genes was analysed using the BLAST program (supra). The full-length ORF has 28% identity and 42% similarity to the outer membrane protein porin N (OmpN) of E. coli, 27% identity and 40% similarity to OmpU of Vibrio cholerae and 28% identity and 40% similarity to OmpK of Klebsiella pneumoniae. These identities and similarities suggest that this major adhesin of A. hydrophila is an outer membrane protein which shares homology with outer membrane proteins of other members of Enterobacteriaceae and Vibrionaceae.

Expression of ahma Gene in E. coli

A. hydrophila major adhesin gene ahma was expressed in E. coli using the QIAexpressionist™ system (QIAGEN) according to instructions recommended by the manufacturer. Briefly, two primers were designed according to the N-terminal and C-terminal of the sequence corresponding to the mature protein and containing BamHI or HindIII restriction enzyme digestion sites: FPQE1: 5′-GCG CGC GGA TCC GCA GTG GTT TAC GAC-3′ and FPQE2: 5′-GCG CGC AAG CTT AGA AGT TGT ATT GCA-3′. These two primers were used for PCR using plasmid pTAH as template. The resulting DNA fragment was recovered from agarose gel by gel purification method. The purified DNA fragment was double-digested by BamHI and HindIII and the resulting fragment was re-purified from the agarose gel. Then, the digested DNA fragment was ligated to the BamHI and HindIII-digested pQE-30 vector and transformed into E. coli M15. The resulting construct was designated as pQE-ahma. Its sequence was confirmed again by DNA sequencing.

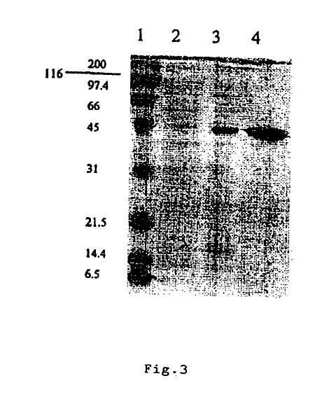

The overnight culture of E. coli M15 harbouring pQE-ahma was diluted 1:20 in fresh LB medium containing 100 μg/Mlle ampicillin and 15 μg/mL kanamycin and grown at 37° C. with vigorous shaking. When OD₆₀₀ of the bacterial broth reached 0.5, isopropyl-1-thio-β-D-galactoside (IPTG) was added to a final concentration of 1 mM. Bacteria were harvested 3 hours after the addition of IPTG by centrifugation at 4800×g at 4° C. for 10 minutes. The bacteria were resuspended in phosphate-buffered saline (PBS, pH 7.4), and 10 μL of cell suspension was boiled in SDS-sample buffer for 5 min and analysed by SDS-polyacrylamide gel electrophoresis (SDS-PAGE). As shown in FIG. 3, the expression of recombinant protein can be induced after addition of IPTG (lane 3). The ahma recombinant protein was purified by 6×His affinity chromatography using Ni-NTA agarose provided in the QIAexpressionist Kit (QIAGEN, USA). Briefly, 500 mL of bacterial culture after IPTG induction was harvested by centrifugation and resuspended in 10 mL of lysis buffer (8M Urea, 100 mM NaH₂PO₄, 10 mM Tri-HCl, pH 8.0). The tube was immersed in ice and the cells were lysed using a sonicator with a 5-mm-diameter probe for 6×30 sec. The lysate was centrifuged at 14,000×g at 4° C. for 20 min. The supernatant was incubated with 2.5 mL of 50% Ni-NTA Superflow™ slurry and mixed gently by shaking at 200 rpm at room temperature for 30 min. The mixture was carefully loaded into an empty column and washed twice with 20 mL of wash buffer (8M Urea, 100 mM NaH₂PO₄, 10 mM Tri-HCl, pH 6.3). The recombinant protein was eluted with 4×1 mL elution buffer D (8M Urea, 100 mM NaH₂PO₄, 10 mM Tri-HCl, pH 5.9), followed by 4×1 mL of buffer E (8M Urea, 100 mM NaH₂PO₄, 10 mM Tri-HCl, pH 4.5). The different eluate fractions were analysed by SDS-PAGE. The fractions containing the protein of interest were pooled together and dialysed against buffer F (1 M Urca, 100 mM NaH₂PO₄, 10 mM Tri-HCl, pH 7.4), followed by PBS (pH 7.4). SDS-PAGE of the pooled fractions that the purified recombinant protein migrated as a single band of about 43-kDa (FIG. 3, lane 4).

Analysis of Antigenic Conservation of 43-kDa Protein

The recombinant adhesin was used to immunise rabbits to obtain anti-recombinant adhesin antisera. For Western Blot, the whole-cell lysates of different strains of Aeromonas, Vibrio and Edwardsiella were boiled in SDS sample buffer for 5 min, and analysed by electrophoresis using a SDS-polyacrylamide gel. The resolved protein was electroblotted onto a 0.2 μm nitrocellulose filter. For immunodetection, the filter was blocked in 5% skimmed milk in TTBS (0.1% Tween-20, 10 mM Tris-HCl, 150 mM NaCl, pH 7.2) at 25° C. for 2 hours, followed by incubating with 1:2000 primary antibody (rabbit antisera against recombinant adhesin protein) at 25° C. for 1.5 hours. After three washes with TTBS, the filter was incubated with 1:50,000 diluted goat anti-rabbit IgG alkaline phosphate conjugates (Bio-Rad) for 1 hour. The filter was washed with TTBS and incubated with substrate NBT (nitroblue tetrazolium) and BCIP (5-bromo-4-chloro-3-indolyl phosphate) for colour development at room temperature.

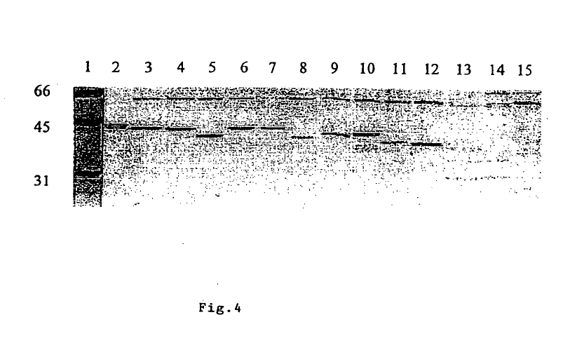

As shown in FIG. 4, the rabbit antisera reacted strongly with two protein bands (with apparent molecular weights of about 43-kDa and 62 kDa, respectively), from all strains of Aeromonas tested, with one protein band (with apparent molecular weights of about 30-kDa) from V. anguillarum, and with one protein band (with apparent molecular weights of about 62-kDa) from Edwardsiella tarda. These results indicate that this 43-kDa adhesin is conserved across Aeromonas species, and that V. anguillarum and E. tarda express proteins with similar antigenic determinants to those of the adhesin of the present invention.

Immunisation of Blue Gourami with Recombinant Adhesin

Blue gourami, Trichogaster trichopterus (Pallas), weighing 10-12 g and measuring 5-8 cm in length were injected intraperitoneally with 15 μg of recombinant adhesin mixed with Freund's complete adjuvant (FCA). For control group, a mixture of 100 μL phosphate-buffered saline (PBS, pH 7.2) and FCA was similarly injected. A booster injection was given to the same fish three weeks later without FCA.

Determination of Antibody Agglutination Titres

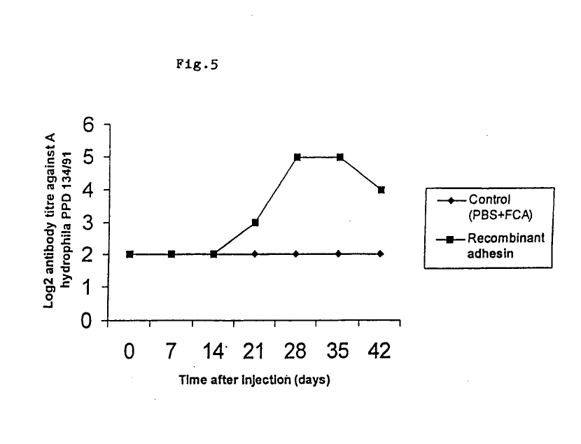

To test the humoral immune response of fish against this recombinant adhesin, the agglutination titre of the antisera obtained from immunised fish was tested against formalin-killed A. hydrophila PPD 134/91 cells. The antibody titres against A. hydrophila PPD 134/91 were determined by agglutination tests in 96-well microtitre plates as described by Roberson (Roberson BS. BACTERIAL AGGLUTINATION. IN: TECHNIQUES IN FISH IMMUNOLOGY. Fair Haven, SOS Publications, 1990, pp. 81-86.). Each week, five fish from immune group or control group were sacrificed to obtain their sera for antibody titre determination. As shown in FIG. 5, the antisera can agglutinate the bacterial cells at high concentration.

Challenge Test with Virulent Strains of A. hydrophila, Vibrio anguillarum and Edwardsiella tarda

To test the effectiveness of the recombinant adhesin as a vaccine, naive blue gourami were immunised with the recombinant adhesin and then challenged with virulent strains of A. hydrophila, V. anguillarum and E. tarda. In this respect, healthy blue gourami were immunised with recombinant adhesin as described in “Immunisation of blue gourami with recombinant adhesin”. Two weeks after the booster injection, the immune and control fish were challenged with live bacteria. These include A. hydrophila PPD 134/91, PPD 70/91 and L31; Vibrio anguillarium 01/10/93(2) and Edwardsiella tarda PPD 130/91. A volume of 0.1 mL of the live bacterial suspension was intraperitoneally injected into fish. The mortality of the fish and other morphological changes were observed and recorded over a period of seven days.

The results are summarised in Table 1. When the immune fish were challenged with homogeneous strains (PPD 134/91), the survival rate of the immunised fish was 95% as compared to 60% for control, giving a relative percent survival (RPS) of 87.5%. When the fish was challenged with a heterologous strain (A. hydrophila PPD 70/91), the RPS was 70%. For another heterologous A. hydrophila L31, the RPS was 28.6%. For a freshwater fish isolated V. anguillarum 01/10/93(2), a RPS of 44.4% was obtained. A similar result was obtained for E. tarda 130/91. These results show that the recombinant adhesin of the present invention can provide protection to blue gourami not only against A. hydrophila, but also against V. anguillarum and E. tarda.

TABLE 1
Extent of protection in Blue gourami immunised with recombinant
adhesin vaccine

| | | | Total | | | |
|---|---|---|---|---|---|---|
| Bacterial strains | Dose | | fish | Dead | Survival | RPS^{b} |
| for challenge | (cells/mL) | Group^{a} | used | Fish | (%) | (%) |
| A hydrophila | 6.0 × 10⁵ | Immune | 20 | 1 | 95 | 87.5** |
| PPD 134/91 | | Control | 20 | 8 | 60 | |
| A hydrophila | 6.1 × 10⁵ | Immune | 20 | 3 | 85 | 70.0** |
| PPD 70/91 | | Control | 20 | 10 | 50 | |
| A hydrophila | 4.45 × 10⁵ | Immune | 20 | 5 | 75 | 28.6 |
| L31 | | Control | 20 | 7 | 65 | |
| V. anguillarum | 1 × 10⁶ | Immune | 20 | 10 | 50 | 44.4* |
| 01/10/93(2) | | Control | 20 | 18 | 10 | |
| E. tarda | 3.28 × 10⁶ | Immune | 20 | 5 | 75 | 44.4 |
| PPD 130/91 | | Control | 20 | 9 | 55 | |

^{a}Experiment had been repeated twice. For immune group, fish were injected with 15 μg of recombinant adhesin in FCA; For control. Fish were injected with PBS and FCA only.
^{b}Significance was tested by Chi-square analysis: **p ≦ 0.01; *p ≦ 0.05

1. A recombinant polypeptide selected from the group consisting of:
(a) a recombinant polypeptide comprising the sequence of SEQ ID NO: 2, 4 or 8;
(b) an immuno-interactive fragment of (a) with the proviso that the fragment does not consist of the sequence of SEQ ID NO: 9;
(c) a variant of (a) or (b);
(d) a derivative of any one of (a) to (c).

2. The polypeptide of claim 1 wherein each of said variant elicits an immune response.

3. The polypeptide of claim 1 wherein said variant of the polypeptide is at least 75% homologous to said polypeptide.

4. The polypeptide of claim 1 wherein said variant of the fragment is at least 75% homologous to said fragment

5. The polypeptide of claim 1 wherein each of said derivative elicits an immune response.

6. The polypeptide of claim 1 wherein the fragment comprises at least six amino acids.

7. A recombinant polypeptide according to claim 1 wherein said recombinant polypeptide comprises the sequence of SEQ ID NO: 8.

8. The polypeptide of claim 7 wherein said polypeptide further comprises a leader peptide comprising the sequence of SEQ ID NO: 6, or a fragment thereof, or variant, or derivative of these.

9. The polypeptide of claim 8 which comprises the sequence of SEQ ID No. 2 or 4.

10. An isolated polynucleotide encoding a polypeptide according to claim 1.

11. The polynucleotide of claim 10 which comprises the sequence of SEQ ID NO: 7.

12. The polynucleotide of claim 1 wherein said polynucleotide further comprises a nucleotide sequence encoding a leader peptide.

13. The polynucleotide of claim 12 wherein said nucleotide sequence comprises the sequence of SEQ ID NO:5, a fragment or a polynucleotide variant thereof.

14. The polynucleotide of claim 13 which comprises the SEQ ID NO: 1 or 3.

15. An isolated polynucleotide comprising the sequence of SEQ ID NO: 1, SEQ ID NO:3, SEQ ID NO:5 or SEQ ID NO:7.

16. An isolated variant of a polynucleotide according to claim 15.

17. The variant of claim 16, said variant comprising a polynucleotide which hybridises to the polynucleotide according to claim 15, or to the complement thereof, under high stringency conditions.

18. The variant of claim 17 which is obtained from a bacterial species.

19. The variant of claim 18 wherein the bacterial species is of a genus selected from the group consisting of Aeromonas, Vibrio and Edwardsiella.

20. The variant of claim 18 wherein the bacterial species is of the genus Aeromonas.

21. An expression vector comprising a polynucleotide of claim 10 wherein the polynucleotide is operably linked to one or more regulatory nucleic acids.

22. A host cell containing the expression vector of claim 21.

23. A method of producing a recombinant polypeptide, comprising:
(a) culturing a host cell containing the expression vector of claim 21; and
(b) isolating the recombinant polypeptide.

24. A method of producing an immuno-interactive fragment, comprising:
(a) producing a fragment of a polypeptide comprising the sequence of SEQ ID NO:2, 4 or 8;
(b) administering said fragment to an animal; and
(c) detecting an immune response in said animal including the production of elements that protect said animal against infection by a bacterial species of a genus selected from the group consisting of Aeromonas, Vibrio and Edwardsiella or that specifically bind to said polypeptide or to said bacterial species, which is indicative of said fragment being said immuno-interactive fragment.

25. The method of claim 24 wherein said elements are antibodies.

26. A method of producing an immuno-interactive fragment, comprising:
(a) combining a fragment of a polypeptide comprising the sequence of SEQ ID NO: 2, 4 or 8 with an antigen binding molecule that binds to said polypeptide; and
(a) detecting the presence of a conjugate comprising said fragment and said antigen-binding molecule wherein said detection is indicative of said fragment being said immuno-interactive fragment.

27. The method of claim 26 wherein the antigen binding molecule is an antibody.

28. The method of claim 27 wherein the presence of said conjugate is detected by an immunoassay.

29. A method of producing a variant of a polypeptide comprising the sequence of SEQ ID NO: 2, 4 or 8 or of an immuno-interactive fragment of said polypeptide, comprising:—
(a) administering a test polypeptide suspected of being a variant to an animal, wherein said test polypeptide is distinguished from said polypeptide or said immuno-interactive fragment by substitution of at least one amino acid with a different amino acid; and
(b) detecting an immune response in said animal, including the production of elements that protect said animal against infection by a bacterial species of a genus selected from the group consisting of Aeromonas, Vibrio and Edwardsiella or that specifically bind to said polypeptide, said immuno-interactive fragment or to said bacterial species, which is indicative of said test polypeptide being said variant.

30. The method of claim 29 wherein said elements are antibodies.

31. A method of producing a variant of a polypeptide comprising the sequence of SEQ ID NO: 2, 4 or 8, or of an immuno-interactive fragment of said polypeptide, comprising:
(a) combining a test polypeptide suspected of being a variant with at least one antigen-binding molecule that binds to said polypeptide or said immuno-interactive fragment, wherein said polypeptide is distinguished from said parent polypeptide or said immuno-interactive fragment by substitution of at least one amino acid with a different amino acid; and
(b) detecting the presence of a conjugate comprising said test polypeptide and said antigen-binding molecule, which is indicative of said test polypeptide being said variant.

32. The method of claim 31 wherein the antigen binding molecule is an antibody.

33. The method of claim 32 wherein the presence of said conjugate is detected in an immunoassay.

34. A composition comprising a polypeptide according to claim 1, and a pharmaceutically acceptable carrier.

35. The composition of claim 34 further comprising an adjuvant.

36. A vaccine comprising the composition of claim 35.

37. A method for eliciting an immune response in an animal, comprising administering to said animal an immunogenically effective amount of a composition comprising a polypeptide according of claim 1 and a pharmaceutically acceptable carrier.

38. The method of claim 37 wherein said immune response includes production of elements that protect said animal against infection by a bacterial species of a genus selected from the group consisting of Aeromonas, Vibrio and Edwardsiella or that specifically bind to said polypeptide or to said bacterial species.

39. The method of claim 38 wherein said elements are antibodies.

40. The method of claim 37 wherein said animal is a freshwater and/or marine animal.

41. The method of claim 37 wherein said animal is a fish.

42. The method of claim 41 wherein said amount is about 1.5 to about 3.0 ug per gram body weight of said fish.

43. The method of claim 41 wherein said amount is about 1.5 ug per gram of body weight of said fish.

44. The method of claim 38 wherein said infection is associated with a condition selected from the group consisting of fish motile aeromonad septicemia, Vibriosis and Edwardsiellosis.

45. The method of claim 37 wherein said composition is administered intraperitoneally.

46. The method of claim 37 wherein said composition is administered by immersion of the animal in said composition.

47. The method of claim 37, wherein said composition is administered by spraying the animal with said composition.

48. An antigen-binding molecule that binds specifically to a polypeptide according to any one of claims 1 to 9.

49. The antigen-binding molecule of claim 48 which is an antibody.

50. A method of detecting in a sample a polypeptide according to any one of claims 1 to 9, comprising:
(a) contacting the sample with the antigen-binding molecule of claim 48; and
(b) detecting the presence of a complex comprising said antigen-binding molecule and the polypeptide in said contacted sample.

51. The method of claim 50 wherein the antigen-binding molecule is an antibody.

52. The method of claim 51 wherein the presence of said complex is detected in an immunoassay.

53. An oligonucleotide which specifically hybridizes to a polynucleotide according to to any one of claims 10 to 20 or its complement.

54. A method of detecting a bacterial species of the genus Aeromonas, Vibrio or Edwardsiella in a biological sample suspected of containing said bacteria, said method comprising:
(a) isolating the biological sample from an animal;
(b) detecting a polynucleotide according to claim 15 or its variant in said sample, which indicates the presence of said bacteria.

55. The method of claim 54 wherein said bacterial species is of the genus Aeromonas.

56. The method of claim 55 wherein said step of detection comprises contacting said sample with an oligonucleotide of claim 53 and detecting whether said oligonucleotide has specifically hybridized to said polynucleotide.

57. The method of claim 55 wherein the step of detection comprises contacting said sample with a first and second oligonucleotide of claim 53 wherein said first and second oligonucleotide are amplification primers, amplifying a nucleotide sequence from said polynucleotide and detecting the amplified sequence.

58. A method of detecting or diagnosing infection of animals by a bacteria comprising:
(a) contacting a biological sample from an animal with a polypeptide according to claim 1; and
(b) determining the presence or absence in said sample of a complex between said polypeptide and antibodies specific to said polypeptide, wherein the presence of said complex is indicative of said infection.

59. The method of claim 58 wherein the bacteria is of the genus Aeromonas, Vibrio or Edwardsiella.

60. A kit for the detection or diagnosis of bacterial infection in an animal wherein said infection is caused by bacteria of the genus Aeromonas, Vibrio, or Edwardsiella, comprising an oligonucleotide according to claim 53.

61. The kit of claim 60 further comprising a polynucleotide according to claim 15.

62. The kit of claim 60 wherein the infection is caused by bacteria of the genus Aeromonas.

63. A kit for the detection or diagnosis of bacterial infection in an animal comprising a polypeptide according to claim 1.

64. The kit of claim 63 wherein said infection is caused by bacteria of the genus Aeromonas, Vibrio, or Edwardsiella.

65. A kit for the detection or diagnosis of bacterial infection in an animal comprising an antigen binding molecule of claim 48.

66. The kit of claim 65 wherein the antigen binding molecule is an antibody.

67. The kit of claim 66 wherein said infection is caused by bacteria of the genus Aeromonas, Vibrio, or Edwardsiella.
